SEQUENCE LISTING (4 sequences)

SEQ ID NO: 1 - DNA, 1181 bp, synthetic construct
  source 1..1181: mol_type = other DNA
aagggaaaacgggtcagaagggtgaaggtgatctgccaagttcacacaacgtaacaagttctgaatagaaggaaaatcaatcttggtctgttccccttggagagactggccttgaggaaagatggtggccagttgattctgatctttctagaactaattctaggacctttcttttttccatataaagcctcttgcccctacaaaagggattcattgattgattaatccatagaacaaatgtacactgagggtcttctataggcaaggcacttccctgagtgctttgagagacagagagctataagaacacattatccttggctcttaaaattcacagcatacatgccttcccactgtcctaaaatatacactcactttgtcacagttaattaagcaatttaaaaaaatggcttggaaatggaatgatgcctcttgagatgacagacctctcggcatgtctcagcactagggttgggagcactgccatactgctcttgcttttcatagatcctgaggcatcagtggggcgagaggctgtgctgtcctcttcctccttcaggaattcagctgcttgtcccctgtcaggagcccctgccctctgaaaggttactcttcagcctggtggggactctgcagtgaacagagctgcaccatccctctggctttcccaaactcttgctccagggcacttgggctttgagggaagagggacttggtgaagaggggatggcaggtggcctccatgtgctccagacctctcctcctctttgcaggtgcactcctcccagcccagctccagaatggccctgccccccagccccctggccatggaatatgtcaatgactttgacttgatgaagtttgaggtaaagcgggaaccctctgagggccgacctggcccccctacagcctcactgggctccacaccttacagctcagtgcctccttcacccaccttcagtgaaccaggcatggtgggggcaaccgagggcacccggccaggcctggaggagctgtactggctggctaccctgcagcagcagctgggggctggggaggcattggggctgagtcctgaagaggccatggagctgctgcagggtcagggcccagtccctgttgatgggccccatggctactacccagggagcccagaggagacaggagcccagcacgtccag

SEQ ID NO: 2 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 3 - DNA, 992 bp, synthetic construct
  source 1..992: mol_type = other DNA
attcaacaacagattcaaccaccaatatttgcacagctagattaatgttttggagtcagtcagtcatcaggctcattactactggattttctctggttcatgctaataaactagattattgattttaggattttctttgtatccaaggatattctctaagatgtctttataatgtttcatgcttaagctgtcaaccaaaatgacataacaatgagtaatcgttttataatcatgttgaaaggggttaaaaacagcatataaaatgcatcttagcagcttcagagtaacattctcgctgtcacttccctatctgggtggaggtcattcctacctgcctgccatcttccccaggaattaactagctgcatgcacagcttaagacctttgatagtcataaatctgctttccaatatgtttggatggcagaagagctcaaaagacacctcggtctaaataggattttagcacatcaggtgctattacaaagtaaagcaggctgggttgctgtgttggtggtggtgtttattcatcttcccttcgcgtttcatgtgtatgtatgtatgcttctctgcgtatatgcccagctctcagggtgtatggggctggagaatgcatgcgtagactgtgtgtatatgtaaactagaaactgaaccagggaaacaaaatgaactactgtatgcaagaaatatatgaagtgcacccagctgccggcagcaattgctacatgcagtccactgattattacgcgtattttgaagatagtccgggttacagcggttgcgatgctcaggctgtgcccagtaacaacatatatatggaacaggcctgggcagtgaatcagccttatacctgtagttaccctggaaacatgtttaaaagcaaggactctgacttggacatggccctgaatcaatacagccaacctgaatatttcactgaagaaaagcctactttttctcaagtgcagtcgccatcgtattctcaaaaaaaag

SEQ ID NO: 4 - DNA, 1428 bp, synthetic construct
  source 1..1428: mol_type = other DNA
atggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaag

(19) United States
(12) Patent Application Publication
(10) Pub. No.: US 2024/0345082 A1
(43) Pub. Date: Oct. 17, 2024

(54) IDENTIFICATION AND APPLICATION OF SELECTIVE CELL SURFACE MARKERS AND ADDITIONAL METHODS FOR HUMAN RED/GREEN CONE PHOTORECEPTOR PRECURSOR ENRICHMENT

(71) Applicant: Wisconsin Alumni Research Foundation, Madison, WI (US)

(72) Inventors: David Gamm, Madison, WI (US); Praveen Joseph Susai Manickam, Madison, WI (US); Michael Phillips, Madison, WI (US); Elizabeth Capowski, Madison, WI (US)

(21) Appl. No.: 18/587,898

(22) Filed: Feb. 26, 2024

Related U.S. Application Data
(60) Provisional application No. 63/486,903, filed on Feb. 24, 2023.

(51) Int. Cl. G01N 33/569; C12N 5/0793

(52) U.S. Cl. CPC G01N 33/56966; C12N 5/062

(57) ABSTRACT

The present disclosure provides methods for obtaining enriched populations of human red/green cone photoreceptor precursor cells with at least one of ALCAM/CD166-positive ITGA6/CD49f-negative, TNFRSF10B/CD262-negative, or NGFR/CD271-negative cell surface markers, compositions of enriched populations of human red/green cone photoreceptor precursor cells, methods for using said compositions for testing and identifying therapeutic agents specific for retinal degenerative diseases, disorders, injuries, or toxicities, and therapeutic agents specific for retinal degenerative diseases, disorders, injuries, or toxicities identified by these methods.

### CROSS-REFERENCE TO APPLICATION

This application claims priority to U.S. provisional application No. 63/486,903, filed Feb. 24, 2023, the disclosure of which is being incorporated by reference herein in its entirety.

### STATEMENT REGARDING FEDERALLY SPONSORED RESEARCH

This invention was made with government support under EY029890, EY032434, and EY033275 awarded by the National Institutes of Health. The government has certain rights in the invention.

### INCORPORATION BY REFERENCE OF SEQUENCE LISTING PROVIDED ELECTRONICALLY

This application contains a Sequence Listing submitted as an electronic text file named “22-0995-US.xml” having a size in bytes of 9 kb and created on Feb. 26, 2024. The information contained in this electronic file is hereby incorporated by reference in its entirety.

### BACKGROUND OF THE DISCLOSURE

Retinal photoreceptors (PRs) are primary visual sensory neurons, and, therefore, their proper connection and function is critical for vision. Cone photoreceptors, for example, are required for daytime vision, as well as color and high acuity vision. Cones can respond to short (blue cones), medium (green cones) and long (red cones) wavelengths of light, but central high acuity vision relies solely on red and green cones, herein referred to as red/green cones.

Loss of PRs because of disease or damage results in vision loss and irreversible blindness. Retinal degenerative diseases, such as age-related macular degeneration, retinitis pigmentosa, and cone-rod disorders, as well as some traumatic or other eye injuries, lead to an acute or progressive loss of cones. One potential treatment approach is cell replacement therapy with human pluripotent stem cell (hPSC)-derived PRs. The technology now exists to generate retinal cells from human pluripotent stem cells, including mixtures of photoreceptors that predominantly contain rods (which are not required for daytime, color or high acuity vision). Cones can be produced within a mixture of retinal cells in 3-dimensional (3D) organoids, but there are no current methods to selectively isolate cone photoreceptor cells without the use of genetic manipulation (e.g., fluorescent tags). Sorting cones at stages (versus mature stages) could lead to better integration following transplantation. The heretofore unrealized ability to sort for human red/green cone photoreceptor precursors (CPPs) has implications for in vitro studies as well, such as drug discovery or gene therapy testing.

Therefore, there is a need in this art for methods for detecting and sorting red/green cone photoreceptor precursor (CPP) cells.

### BRIEF SUMMARY OF THE DISCLOSURE

Provided herein are methods for obtaining enriched populations of human red/green CPP cells comprising sorting a mixed population of retinal cells contacted with a detectably labeled binding agent that specifically binds ALCAM/CD166 expressed on the cell surface of human red/green CPP cells. In specific embodiments these methods further comprise contacting the cells with the detectably labeled specific binding agent for ALCAM/CD166 before or during sorting; in particular embodiments the specific binding agent is an antibody or antigen-binding fragment thereof that specifically binds ALCAM/CD166. In certain embodiments these methods further comprise sorting the cells by magnetic-activated cell sorting (MACS).

Also provided herein are populations of human red/green cone photoreceptor precursor cells wherein cells comprising the population express at the cell surface ALCAM/CD166. Said populations are advantageously produced by methods for obtaining enriched populations of human red/green CPP cells comprising sorting a mixed population of retinal cells contacted with a detectably labeled binding agent that specifically binds ALCAM/CD166 expressed on the cell surface of human red/green CPP cells. In specific embodiments these methods further comprise contacting the cells with the detectably labeled specific binding agent for ALCAM/CD166 before or during sorting; in particular embodiments the specific binding agent is an antibody or antigen-binding fragment thereof that specifically binds ALCAM/CD166. In certain embodiments these methods further comprise sorting the cells by magnetic-activated cell sorting (MACS).

Further provided herein are methods for identifying therapeutic agents specific for human red/green CPP cells, comprising contacting a population of human CPP cells that express at the cell surface ALCAM/CD166 with one or a plurality of drug candidates and detecting a physiologic response thereto.

Provided herein are methods of treating a disease or disorder in cone photoreceptor cells in an animal, comprising administering to an individual in need thereof a therapeutically effective amount of a therapeutic agent specific for cone photoreceptor cells.

Also provided herein are methods of treating a disease or disorder in cone photoreceptor cells in an animal, comprising administering to an individual in need thereof a therapeutically effective amount of a therapeutic agent wherein the disease or disorder involves cone-rich macula, including but not limited to cone-rod dystrophies, macula-off retinal detachments, laser injuries, commotio retinae, or chloroquine or hydroxychloroquine toxicity or other diseases, injuries or toxicities. In certain embodiments the disease is age-related macular degeneration, myopic degeneration, Stargardt disease, or Best disease.

Additionally, provided herein are methods for detecting red/green CPP cells, the methods comprising identifying CPP cells from a three-dimensional (3D) retinal organoid based on cell surface expression of cell surface marker ALCAM/CD166, wherein the 3D retinal organoid is derived from stem cells and comprises photoreceptor cells and other retinal cell types. In certain embodiments the stem cells are induced pluripotent stem cells (iPSCs) or human embryonic stem cells (hESCs). In further embodiments, the stem cells are dual reporter lines comprising two fluorescent proteins, wherein one fluorescent protein is a red fluorescent protein (RFP), or wherein another fluorescent protein is a green fluorescent protein (GFP). In certain embodiments the RFP is tdTomato. In certain embodiments the fluorescent proteins are encoded by a nucleotide sequence operably linked to a promoter. In particular embodiments the promoter is a thyroid hormone receptor beta 2 (THRB2) promoter. In particular embodiments, the RFP is linked to the THRB2 promotor. In particular embodiments the THRB2-RFP reporter line indicates red/green cone precursors. In certain additional embodiments the fluorescent proteins are encoded by a nucleotide sequence operably linked to a neural retina-specific leucine zipper protein promoter (NRL). In certain embodiments the GFP is linked to the NRL promotor. In certain embodiments, the NRL-GFP reporter line indicates rod precursors.

Provided herein are populations of red/green cone CPP cells from a subject, the population comprising enriched CPP cells, wherein the CPP cells comprising the population expressing at the cell surface ALCAM/CD166. In certain embodiments the subject is a mammal and in particular embodiments the mammal is a human.

Also provided herein are methods for identifying therapeutic agents specific for retinal degenerative diseases or disorders or retinal injuries or toxicities, the methods comprising contacting a population of red/green CPP cells with one or a plurality of drug candidates and detecting a therapeutically beneficial physiologic response thereto.

Additionally provided herein are methods of treating a retinal degenerative disease or disorder or retinal disease or toxicity in a subject, the method comprising administering to a subject in need thereof a therapeutically effective amount of a therapeutic agent; wherein the subject is a mammal; and wherein the mammal is a human.

Further provided herein are methods of treating a retinal degenerative disease or disorder or retinal disease or toxicity in a subject, the methods comprising administering to a subject in need thereof a therapeutically effective amount of a therapeutic agent, wherein the disease or disorder involves cone-rich macula, including but not limited to cone-rod dystrophies, macula-off retinal detachments, laser injuries, commotio retinac, or chloroquine or hydroxychloroquine toxicity or other injuries or toxicities. In certain embodiments the disease is age-related macular degeneration, myopic degeneration, Stargardt disease, or Best disease.

Also provided herein are methods of obtaining an enriched population of red/green CPP cells, the method comprising sorting a mixed population of retinal cells contacted with one or more of detectably labeled binding agents each of which specifically binds ITGA6/CD49f, TNFRSF10B/CD262, NGFR/CD271, or combinations thereof, expressed on the cell surface of non-CPP cells, and sorting to obtain ITGA6/CD49f, TNFRSF10B/CD262, NGFR/CD271-negative CPP cells. In specific embodiments, the binding agent is an antibody or antigen-binding fragment each of which specifically binds ITGA6/CD49f, TNFRSF10B/CD262, NGFR/CD271, or combinations thereof. In certain embodiments the antibody is one or more biotin-conjugated antibodies, wherein the biotin-conjugated antibody is one or more of ITGA6/CD49f, TNFRSF10B/CD262, NGFR/CD271, or combinations thereof. In specific embodiments, the cells are incubated with one or more biotin-conjugated antibodies, incubated with anti-biotin microbeads, and then sorted via magnetic-activated cell sorting (MACS). In certain embodiments, the sorted cells are separated into one or more of ITGA6/CD49f-negative, TNFRSF10B/CD262-negative, NGFR/CD271-negative, or combinations thereof and ITGA6/CD49f-positive, TNFRSF10B/CD262-positive, NGFR/CD271-positive, or combinations thereof cell fractions.

Provided herein are populations of human CPP cells, wherein the CPP cells comprising the population do not express at the cell surface at least one of ITGA6/CD49f, TNFRSF10B/CD262, NGFR/CD271, or combinations thereof. The cells are advantageously produced by the methods of obtaining an enriched population of red/green CPP cells, sorting a mixed population of retinal cells contacted with a detectably labeled binding agent each of which specifically binds ITGA6/CD49f, TNFRSF10B/CD262, NGFR/CD271, or combination thereof expressed on the cell surface, and sorting the cells for ITGA6/CD49f-negative, TNFRSF10B/CD262-negative, NGFR/CD271-negative cells, or combinations thereof. In specific embodiments, the binding agent is an antibody or antigen-binding fragment each of which specifically binds ITGA6/CD49f, TNFRSF10B/CD262, NGFR/CD271, or combinations thereof. In certain embodiments the antibody is one or more biotin-conjugated antibodies, wherein the biotin-conjugated antibody is one or more of ITGA6/CD49f, TNFRSF10B/CD262, NGFR/CD271, or combinations thereof. In specific embodiments, the cells are incubated with the one or more biotin-conjugated antibodies. In further embodiments, the cells are then incubated with anti-biotin microbeads. In certain embodiments these methods further comprise sorting the cells by magnetic-activated cell sorting (MACS). In certain embodiments, the sorted cells are separated into one or more of ITGA6/CD49f-negative, TNFRSF10B/CD262-negative, NGFR/CD271-negative, or combinations thereof and ITGA6/CD49f-positive, TNFRSF10B/CD262-positive, NGFR/CD271-positive, or combinations thereof cell fractions

Also provided herein are methods of obtaining microaggregates from a population of CPP cells. The CPP microaggregates are advantageously produced by the methods for research use or transplantation purposes. The methods comprise seeding at least one of ALCAM/CD166-positive, ITGA6/CD49f-negative, TNFRSF10B/CD262-negative, NGFR/CD271-negative CPP cells, or combinations thereof at a seeding density. In some embodiments, at least one of ALCAM/CD166-positive, ITGA6/CD49f-negative, TNFRSF10B/CD262-negative, NGFR/CD271-negative cells, or combinations thereof, are seeded on agarose microwells. In some embodiments, the seeding density is 250-8000 cells/microwell, and wherein the cells are seeded at a seeding density.

Further provided herein are methods of obtaining CPP cell microaggregates for transplantation. The methods comprise treating a population of CPP cell microaggregates advantageously produced by methods of obtaining microaggregates from a population of CPP cells. In further embodiments the methods include treating the population of CPP cells with a cell proliferation inhibitor and incubating CPP cell microaggregates in 3D-Retinal Differentiation Medium (RDM) containing the cell proliferation inhibitor for an amount of time, wherein cells are washed with 3D-RDM after incubation. In some embodiments the cell proliferation inhibitor is mitomycin C. In some embodiments the CPP cell microaggregates are incubated for 24-72 hours.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents, and alternatives. Reference should therefore be made to the claims recited herein for interpreting the scope of the invention.

### BRIEF DESCRIPTION OF THE DRAWINGS

The disclosure will be better understood and features, aspects, and advantages other than those set forth above will become apparent when consideration is given to the following detailed description thereof. Such detailed description refers to the following figures.

FIG. 1 is a model of a human retina. Two types of photoreceptor cells (rod and cone cells) in the retina convert light to nerve impulses that are ultimately sent to the brain enabling vision. Rod photoreceptors are required for peripheral and night vision. Cone photoreceptors—and particularly red-green cones as mentioned above—are required for central and color vision.

FIG. 2 shows a model of stem cells being used as a source to grow retinal organoids. A schematic view of the organoid (right panel) illustrates that these organoids predominantly contain rod photoreceptors with sparser populations of cone photoreceptors therein.

FIG. 3 is a schematic diagram of two genomic loci in the same cell line (the cone rod reporter (CRR) line) incorporating fluorescent reporter genes on one chromosome at each locus. The THRB2-tdTomato reporter labels specifically cone photoreceptors and the NRL-cGFP reporter labels specifically rod photoreceptors.

FIG. 4A-FIG. 4B show expression of the fluorescent reporter proteins in cone and rod cells of D45 and D100 CRR retinal organoids respectively. More specifically, THRB2 and NRL are intracellular markers of early red/green cones and rods, respectively. FIG. 4A shows THRB2-tdTomato expression in early red/green cone photoreceptor precursor (CPP) cells and do not show NRL-eGFP expression due to the absence of early rods at this D45 time in development. FIG. 4B shows THRB2-tdTomato expression in late (maturing) cone photoreceptor cells and NRL-eGFP expression in early rod cells at D100, the latter of which begin to arise around D70. Scale bar: 50 μm.

FIG. 5 shows a Venn diagram of 1891 upregulated genes in photoreceptors (early red/green cones, late red/green cones, and rods) versus non-photoreceptor cells. Upregulated genes were identified via bulk RNAseq analysis of FAC-sorted retinal organoid cells derived from the CRR dual reporter line (D50 non-photoreceptors (D50 N), D50 red/green cones (D50 Th-C), D100 non-photoreceptors (D100 N), D100 red/green cones (D100 Th-C), D100 rods (D100 R)). The 313 genes (yellow box) solely expressed in D50 red/green cones are shown in the form of a heat map in FIG. 6A.

FIG. 6A-FIG. 6B show heatmaps of gene expression in red/green cone CPPs. FIG. 6A shows a heat map of 313 genes expressed solely in D50 early red/green cone precursors. (red=higher expression, blue=lower expression). FIG. 6B identifies 5 early red/green CPP-specific cell surface differentiation (CD) markers present within those 313 genes, which includes ALCAM/CD166 (highlighted).

FIG. 7 is a photomicrograph showing ALCAM/CD166 expression in D50 retinal organoids from a second (non-CRR reporter) human pluripotent stem cell line (WA09). Scale bar: 50 μm.

FIG. 8A-FIG. 8F show photomicrographs of ALCAM/CD166 expression in D50—but not D100—WA09 retinal organoids, confirming the RNAseq results from the CRR retinal organoids. FIG. 8A is a merged image of all fluorescent channels (insets magnified in FIG. 9), FIG. 8B shows DAPI-stained nuclei specific for nucleic acids (blue). FIG. 8C shows the presence of proliferating KI67-positive cells (green) in D50 and D100 organoids, FIG. 8D shows the presence of ALCAM/CD166 positive cells only in D50 organoids and not in D100 organoids (red), FIG. 8E shows the presence of Recoverin (RCVRN)-positive photoreceptors (purple) in both D50 and D100 organoids. Note that RCVRN-positive photoreceptors increase in number over time. FIG. 8F shows expression of ALCAM/CD166 in RCVRN-positive/KI67-negative post-mitotic CPPs in cells deep within the organoid in a layer corresponding to newborn postmitotic photoreceptors. Scale bar: 100 μm.

FIG. 9A-FIG. 9E show photomicrographs of the higher magnification boxed inset from FIG. 8. For better visualization, the pseudocolors of KI67 (purple) and RCVRN (green) are exchanged. FIG. 9A shows KI67 immunostaining in purple marking proliferative cells in both D50 and D100 retinal organoids. FIG. 9B shows ALCAM/CD166 immunostaining in red marking the presence of ALCAM/CD166-positive CPPs only in D50 organoids and not in D100 organoids. FIG. 9C shows RCVRN immunostaining in green showing the increasing presence of photoreceptors in D100 vs. D50 organoids. FIG. 9D shows three fluorescent channels merged to show expression of ALCAM/CD166 within RCVRN-positive/KI67-negative post-mitotic CPPs, (Scale bar: 200 μm). FIG. 9E shows a magnified inset from FIG. 9D illustrating the absence of KI67 immunostaining in post-mitotic ALCAM/CD166-positive/RCVRN-positive CPPs.

FIG. 10 shows photomicrographs illustrating enrichment of CPP cells following ALCAM/CD166-based magnetic activated cell sorting (MACS) from dissociated CRR D50 retinal organoids. Scale bars: 50 μm.

FIG. 11A-FIG. 11C show quantification of THRB:tdTomato-positive red/green CPP cells by flow cytometry (FC) in CRR retinal organoids at D50 after ALCAM/CD166-based MACS. FIG. 11A shows the percentage of THRB:tdTomato-positive red/green CPP cells in unsorted and ALCAM/CD166 based MAC-sorted positive and negative fractions. Initial MACS was performed using the manufacturer's recommended ALCAM/CD166 dilution of 1 in 50. FIG. 11B shows the percentage of THRB2:tdTomato-positive red/green CPP cells in the positive and negative fraction after ALCAM/CD166 based MACS using different dilutions of ALCAM/CD166-1 in 50 (blue box), 1 in 250 (purple box) and 1 in 500 (pink box). Black box-No fluorescence control, Brown box-Dissociated and unsorted CRR retinal organoid cells at D50. FIG. 11C shows a bar graph of the percentage of THRB:tdTomato-positive red/green CPP cells in unsorted, ALCAM/CD166-positive and ALCAM/CD166-negative cells after MACS using ALCAM/CD166 at different dilutions. It can be noted that there is an increase in the percentage of THRB2:tdTomato-positive red/green CPP cells with increased dilution.

FIG. 12 shows a heat map of 26 CD markers that can be used for negative sorting of red/green CPP cells.

FIG. 13A-FIG. 13B show flow cytometric quantification of THRB2:tdTomato-positive red/green CPP cells from CRR D50-D56 ROs obtained by MACS negative sorting compared to unsorted or CD166-sorted ROs. FIG. 13A shows the percentage of THRB2:tdTomato-positive red/green CPP cells in unsorted ROs (13A.i), the CD166-positive fraction of CD166-sorted ROs (FIG. 13A.ii), and the negative fractions of CD49f-, CD262-, or CD271-sorted ROs when used individually (13A.iv-13A.vi) or in combinations (13A.vii-13A.x). Negative MACS was performed using the manufacturer's recommended dilution. FIG. 13B shows a bar graph of the percentage of THRB2:tdTomato-positive red/green CPP cells obtained by negative MACS (using individual antibodies or in specific combinations) in comparison to unsorted ROs and the CD166+ fraction of CD166-sorted ROs (antibody dilution-1 in 50).

FIG. 14 shows a diagram of CPP microaggregate preparation using ALCAM/CD166-based MACS of D50 CRR retinal organoids. The same method was used to prepare WA09 CPP microaggregates used for the rat transplantation experiment shown in FIG. 17. FIG. 14.i shows CPP microaggregates in individual agarose microwells and expressing tdTomato. Scale bar: 200 μm.

FIG. 15 shows a diagram of mitomycin C (MMC) treatment of ALCAM/CD166-based MAC-sorted cells during CPP microaggregates formation and ICC images of sectioned microaggregates 7 days post treatment (dpt) of both control (FIG. 15.ii-iii) or MMC-treated CPP microaggregates (FIG. 15.iv-15.vi). Scale bars: FIG. 15.i—200 μm, FIG. 15.ii-vi—50 μm.

FIG. 16 shows flow cytometric quantification of THRB2:tdTomato-positive red/green CPP cells in CPP microaggregates treated with DMSO (control) or MMC (250 ng/ml) 10 days post microaggregate formation (dpbf) in comparison to unsorted CRR ROs at D60.

FIG. 17A-FIG. 7C show photomicrographs of IHC analysis of retinal cross-sections of Foxn1-S334ter rats. This immunocompromised rat model has a rhodopsin mutation that leads to rapid rod photoreceptor loss and progressive cone loss, similar to patients with retinitis pigmentosa. At 2 months of age, rods are essentially lost, and cone degeneration is underway. Rats were injected at 2 months of age with unsorted CPP microaggregates from the WA09 human embryonic stem cell line treated with DMSO (FIG. 17A) or CPP microaggregates treated with 250 ng/mL MMC (FIG. 17B) and followed for 3 months. Eyes were collected for histology. Age-matched uninjected Foxn1-S334ter rat eyes served as controls (FIG. 17C). Scale bars: 50 μm.

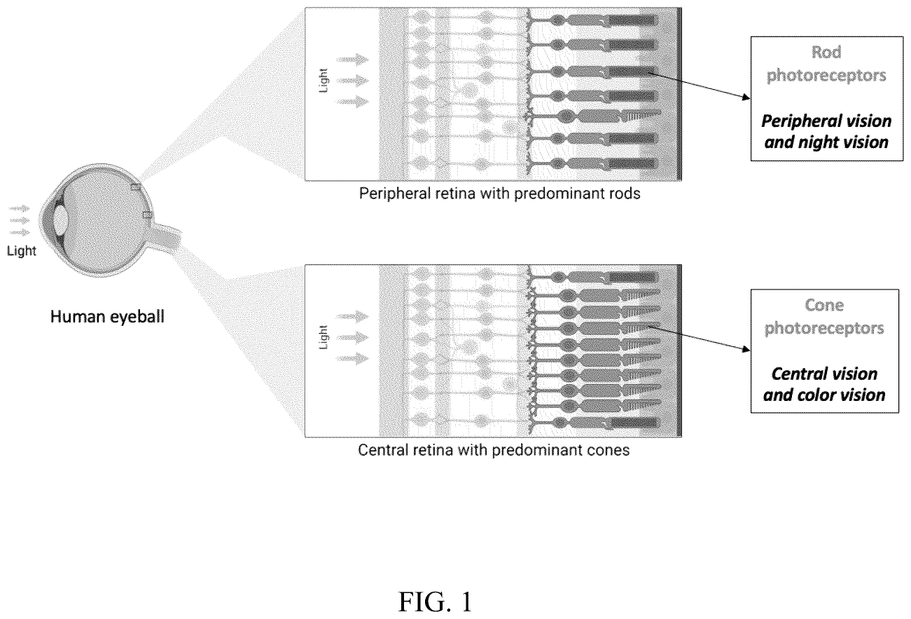

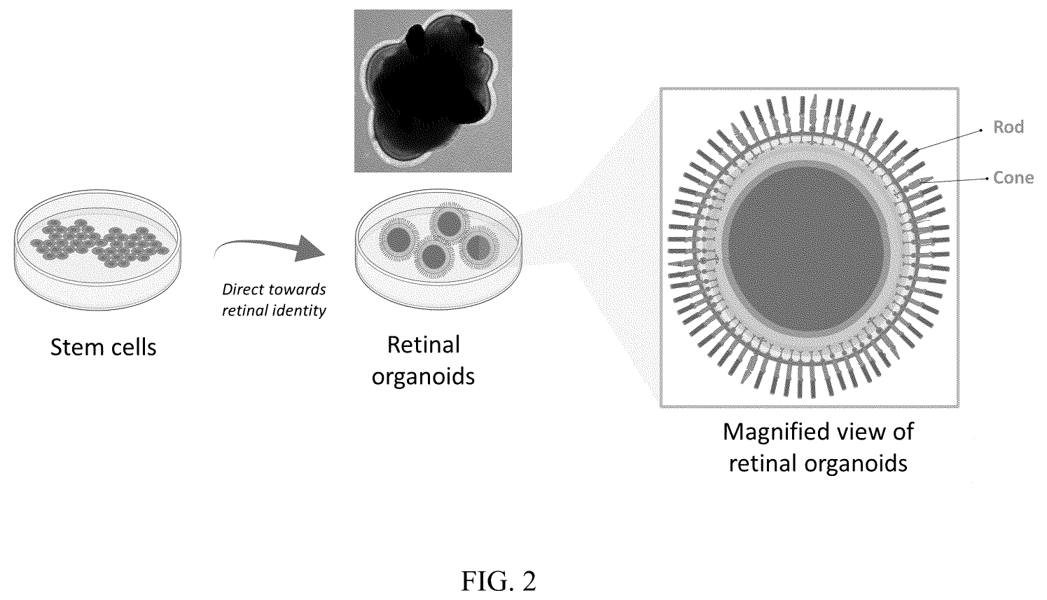

### DETAILED DESCRIPTION OF THE DISCLOSURE

For the purposes of explicating and understanding the principles of this disclosure, reference is made to embodiments and specific language used to describe the same. The skilled artisan will nevertheless understand that no limitation of the scope of the disclosure is thereby intended, such alteration and further modifications of the disclosure as illustrated herein, being contemplated as would be understood by one skilled in the art to which the disclosure relates.

### Definitions

As used herein, articles “a” and “an” are intended to refer to one or to more than one (i.e., at least one) of the grammatical object of the article. By way of example, “an element” means at least one element and can include more than one element.

“About” is used to provide flexibility to a numerical range endpoint by providing that a given value can be “slightly above” or “slightly below” the endpoint without affecting the therapeutically beneficial result. The term “about” in association with a numerical value means that the numerical value can vary by plus or minus 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements, or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

Recitation of ranges of values herein are merely intended to serve as a succinct method of referring individually to each separate value falling within the range, unless otherwise indicated herein. Furthermore, each separate value is incorporated into the specification as if it were individually recited herein. For example, if a range is stated as 1 to 50, it is intended that values such as 2 to 4, 10 to 30, or 1 to 3, etc., are expressly enumerated in this disclosure. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by a person of ordinary skill in the art to which this disclosure belongs.

The terms “express” or “expression” refer to transcription and translation of a nucleic acid coding sequence resulting in production of the encoded polypeptide. “Express” or “expression” also refers to antigens that are expressed on cell surfaces.

As used herein, the term “subject” refers to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The subject can be a human patient that is at risk for, or suffering from, one or more retinal diseases or disorders. The human subject can be of any age (e.g., an infant, child, or adult).

The term “construct” refers to an artificially designed segment of DNA that can be used to incorporate genetic material into a target cell.

The term “sequence identity” refers to the number of identical or similar nucleotide bases on a comparison between a test and reference oligonucleotide or nucleotide sequence. Sequence identity can be determined by sequence alignment of a first nucleic acid sequence to identify regions of similarity or identity to second nucleic acid sequence. As described herein, sequence identity is generally determined by alignment to identify identical residues. Matches, mismatches, and gaps can be identified between compared sequences by techniques known in the art. Alternatively, sequence identity can be determined without taking into account gaps as the number of identical positions/lengths of the total aligned sequence×100. In one embodiment, the term “at least 90% sequence identity to” refers to percent identities from 90 to 100%, relative to the reference nucleotide sequence. Identity at a level of 90% or more is indicative of the fact that, assuming for exemplary purposes a test and reference polynucleotide sequence length of 100 nucleotides are compared, no more than 10% (i.e., 10 out of 100) of the nucleotides in the test oligonucleotide differ from those of the reference oligonucleotide. Differences are defined as nucleic acid substitutions, insertions, or deletions.

The term “enriched,” as used herein, refers to a population of cells of a particular cell type (e.g. cone photoreceptor cells) that has been isolated or sorted from a mixed cell population.

The term “physiological response,” as used herein, refers to any response relevant to cone photoreceptor survival and function in response to a stimulus, including, but not limited to, effects on light detection, metabolism, signal transduction, and cone survival.

The term “red/green cone photoreceptor precursors (CPPs),” as used herein, refers to a transient, early (before day (d) 100 of differentiation) population of recently post-mitotic red/green cone photoreceptors expressing thyroid hormone receptor beta 2 (THRB2) and not neural retina-specific leucine zipper protein (NRL).

The term “cell surface marker,” as used herein, refers to proteins expressed on the cell surface or carbohydrates attached to cell membranes of specific cell types wherein this expression enables cells expressing the marker or markers to be identified.

The term “cell surface marker sorting technique,” as used herein, refers to any method for separating cells expressing a specific cell surface marker from cells lacking expression of the cell surface marker.

In some embodiments, the cell surface marker sorting techniques includes, but it not limited to, fluorescent activated cell sorting (FACS), magnetic activated cell sorting (MACS), immunoprecipitation, immunodensity cell isolation, and centrifugation.

In specific embodiments, the cell surface marker sorting technique is FACS or MACS.

The term “reporter line” or “reporter cell line,” as used herein, refers to a cell line that can be used to visualize, track, and sort cells in real-time.

The term “blocking solution” as used herein refers to any solution consisting of a protein or compound, or mixture of proteins or compounds, that passively adsorb to non-targeted binding surfaces in a sample subjected to immunocytochemistry (ICC), thus reducing or removing non-specific background signal. In addition to preventing or blocking background interferences (e.g., cellular autofluorescence or endogenous non-specific enzyme activity), blocking solutions prevent non-specific binding of primary and secondary antibodies.

In some embodiments, CPPs are identified by cell surface marker expression for a 3-dimensional (3D) retinal organoid.

In some embodiments, the 3D retinal organoid is derived from stem cells.

In some embodiments, the stem cells are induced pluripotent stem cells (iPSCs) or human embryonic stem cells (hESCs).

In further embodiments, the 3D retinal organoid is labeled with a detectable label.

As used herein, the terms “detectable label” and “detectably labeled” is intended to encompass molecules that can be detected due to having a physical property, including but not limited to fluorescence when illuminated at a characteristic wavelength, having a characteristic color when illuminated by light in the visible spectrum, magnetic properties that respond to a magnetic field.

In some embodiments, the detectable label is a fluorescent protein.

In some embodiments, the fluorescent protein is a red fluorescent protein (RFP).

In exemplary embodiments, the RFP is tdTomato.

In other embodiments, the fluorescent protein is a green fluorescent protein (GFP).

In some embodiments, the fluorescent protein is regulated by a promoter.

In some embodiments, the promoter is the human thyroid hormone receptor beta gene (THRB2) promoter.

In specific embodiments, the promoter is the neural retina-specific leucine zipper gene (NRL) promoter.

In further embodiments, the expression of GFP under the NRL promoter indicates rod precursors.

In even further embodiments, tdTomato expressed under the THRB2 promoter indicates red/green cone precursors.

In some embodiments, RNAseq analysis is used to detect cell surface markers in red/green cone precursors.

In exemplary embodiments, the cell surface marker is ALCAM/CD166, which is transiently expressed in red/green CPP cells.

As used herein, the term “positive sorting” refers to CD166+ CPP cells that are retained in the column using MACS using the disclosed methods.

As used herein, the term “negative sorting” refers to CPP cells that are sorted using MACS to remove non-CPP cells, wherein CPP cells are not retained in the column using the disclosed methods. Typically, the CPP cells remain in the flowthrough eluate after MACS.

Enriched populations of red/green CPP cells can be obtained by positive MACS sorting, negative MACS sorting, or a combination of negative and positive MACS sorting.

As used herein, the term “specific binding agent” is intended to encompass any molecule each of with specifically binds to ALCAM/CD166, ITGA6/CD49f, TNFRSF10B/CD262, NGFR/CD271, or combinations thereof. In exemplary embodiments the specificity of such molecules is sufficient to have low, minimal, or no binding specificity for other molecules expressed on the cell surface of cells in a mixture with cells expressing cell surface ALCAM/CD166, ITGA6/CD49f, TNFRSF10B/CD262, NGFR/CD271, or combinations thereof. In particular embodiments the invention provides methods for using specific binding agents ALCAM/CD166, ITGA6/CD49f, TNFRSF10B/CD262, NGFR/CD271, or combinations thereof that are antibodies of ALCAM/CD166, ITGA6/CD49f, TNFRSF10B/CD262, NGFR/CD271, or combinations thereof of antigen-binding fragments thereof. Said antibodies can be produced by immunizing an animal, as monoclonal antibodies prepared as understood in the art, or by recombinant genetic methods. In certain embodiments ALCAM/CD166, ITGA6/CD49f, TNFRSF10B/CD262, NGFR/CD271, or combinations thereof antibodies or ALCAM/CD166, ITGA6/CD49f, TNFRSF10B/CD262, NGFR/CD271, or combinations thereof of antigen-binding fragments thereof are commercially available (e.g., Miltenyi Biotec-Catalog number: 130-126-100 and BioLegend-Catalog number: 343902).

In particular embodiments the specific binding agents are detectably labeled as disclosed herein. In further embodiments the specific binding agent is an antibody or antigen-binding fragment thereof that specifically binds ALCAM/CD166.

In exemplary embodiments cells expressing ALCAM/CD166 are contacted with a specific binding agent that specifically binds to ALCAM/CD166 expressed at the cell surface.

In further embodiments, ALCAM/CD166 positive cells are selected by a using a cell surface marker sorting technique.

In exemplary embodiments, CPP cells are positively-sorted and purified from a mixed population of retinal cells by magnetic activated cell sorting (MACS). The population of human red/green CPP cells express at the cell surface ALCAM/CD166. Purified ALCAM/CD166-positive (ALCAM/CD166+) CPP cells can be used for transplantation therapy to treat cone-specific retinal (or macular) diseases and are more plastic than mature cells.

In some embodiments, cells are positively or negatively sorted using one or more detectably labeled binding agents. The binding agent can be an antibody or antigen-binding fragment.

In exemplary embodiments, the population of cells comprises CPP cells which can be positively-sorted using CD166-specific antibodies. These sorted populations comprise two components, a CD166+ component and a CD166-negative component that also expresses at least one of three other cell surface markers not expressed by CD166+ cells.

In some embodiments the specific binding agent is an antibody or antigen-binding fragment thereof that specifically binds ALCAM/CD166.

In some embodiments the binding agent specifically binds ITGA6/CD49f, TNFRSF10B/CD262, NGFR/CD271, or combinations thereof.

In some embodiments, the antibody is one or more biotin-conjugated antibodies selected from ITGA6/CD49f, TNFRSF10B/CD262, NGFR/CD271, or combinations thereof.

In further embodiments, ITGA6/CD49f-negative, TNFRSF10B/CD262-negative, NGFR/CD271-negative cells, or combinations thereof are negatively-sorted.

In some embodiments, the ITGA6/CD49f-negative, TNFRSF10B/CD262-negative, NGFR/CD271-negative cells, or combinations thereof, are incubated with anti-biotin microbeads. The cells can be sorted by MACS. In some embodiments, ITGA6/CD49f-negative, TNFRSF10B/CD262-negative, NGFR/CD271-negative cells, or combinations thereof, and ITGA6/CD49f-positive, TNFRSF10B/CD262-positive, NGFR/CD271-positive cells, or combinations thereof, are separated into fractions. Accordingly, CPP can be sorted to provide an enriched population of cells using magnetic microbeads conjugated to at least one of the three cells surface markers not expressed on CPP cells wherein, ITGA6/CD49f-positive, TNFRSF10B/CD262-positive, NGFR/CD271-positive cells, or combinations thereof, cells are retained in the column but are not in the flowthrough.

In some embodiments the population of human red/green CPP cells do not express at the cell surface any of ITGA6/CD49f, TNFRSF10B/CD262, NGFR/CD271 cells, or combinations thereof. Purified ITGA6/CD49f-negative, TNFRSF10B/CD262-negative, NGFR/CD271-negative cells, or combinations thereof cells can be used for transplantation therapy to treat cone-specific retinal (or macular) diseases and are more plastic than mature cells.

The term “microaggregate” refers to CPP microspheroids formed on agarose microwells.

In some embodiments, microaggregates are obtained from the population of positive- or negative-sorted CPP cells. ALCAM/CD166-positive, ITGA6/CD49f-negative, TNFRSF10B/CD262-negative, NGFR/CD271-negative cells, or combinations thereof can be seeded, inter alia, on agarose microwells. In some embodiments, the ALCAM/CD166-positive, ITGA6/CD49f-negative, TNFRSF10B/CD262-negative, NGFR/CD271-negative cells, or combinations thereof are seeded for at least 24 hours.

In further embodiments at least one of ALCAM/CD166-positive, ITGA6/CD49f-negative, TNFRSF10B/CD262-negative, NGFR/CD271-negative cells, or combinations thereof are seeded on agarose microwells.

In yet further embodiments, the seeding density is 250-8000 cells/microwell.

In some embodiments CPP microaggregates are treated with a cell proliferation inhibitor.

In certain embodiments the cell proliferation inhibitor is mitomycin C.

In further embodiments CPP microaggregates are incubated in 3D-Retinal Differentiation Media (RDM) containing the cell proliferation inhibitor.

In some embodiments the microaggregates are incubated for 24-72 hours.

CPPs lead to better and more consistent photoreceptor layer reconstruction and integration following transplantation. Additionally, CPPs have optimal plasticity compared to more mature cones (Rempel et al., 2022, Cell Rep. 39 (7): 110827). CPPs can be used for in vitro studies as well.

In some embodiments, a therapeutic agent or agents for treating a red/green cone photoreceptor disease or disorder in an animal are identified by contacting a population of human red/green CPP cells with one or a plurality of drug candidates and detecting a physiologic response thereto.

In some embodiments, the therapeutic agents treat a disease or disorders wherein the disease or disorder affects the red/green cone-rich macula.

In exemplary embodiments, the disease or disorder is any disease or disorder that leads to degeneration or death of the red/green cone-rich macula, including, but not limited to, age-related macular degeneration, Best disease, Stargardt disease, myopic degeneration, cone-rod dystrophies, macula-off retinal detachments, laser injuries, commotion retinae, chloroquine or hydroxychloroquine toxicity, or any other macular disease, injury, or toxin-induced cone dysfunction or degeneration.

Various exemplary embodiments of compositions and methods according to this invention are now described in the following non-limiting Examples. The Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

The Examples set forth herein incorporate and rely on certain experimental and preparatory methods and techniques preformed as exemplified herein.

### Example 1

### Generation of Cone Rod Reporter (CRR) Cell Line

A reporter cell line, NRL-eGFP, was created as reported in Phillips and Capowski et al. (2018 Sci Rep. 8 (1): 2370). Briefly, the eGFP gene (SEQ ID NO:2) was amplified from the Addgene plasmid 80945 by polymerase chain reaction (PCR) and introduced into cellular genomic DNA by CRISPR-Cas9 based gene editing, replacing the original NRL gene that was under the control of the NRL promoter.

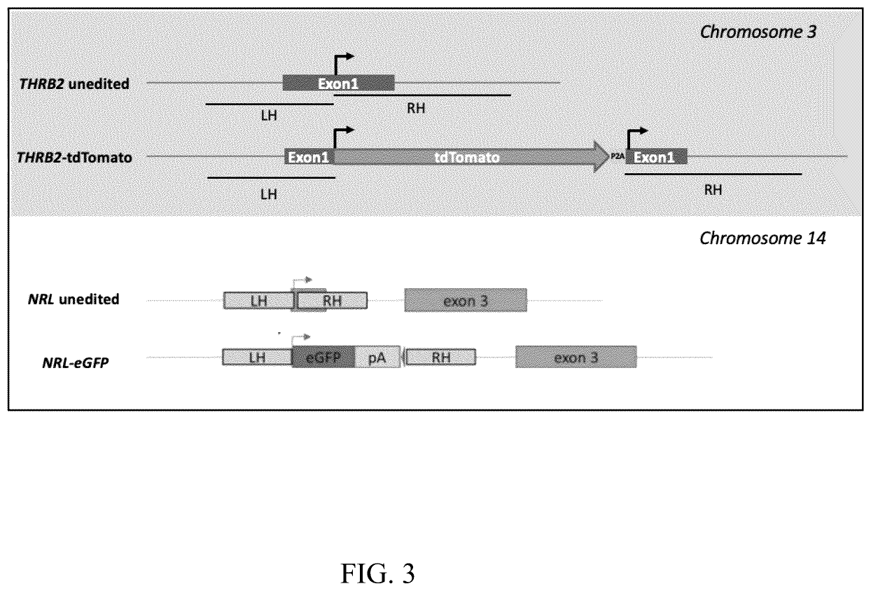

The cone rod reporter (CRR) line was created by adding another fluorescent reporter, tdTomato, to the NRL-reporter line. Briefly, the tdTomato gene (SEQ ID NO: 4) minus a stop codon was amplified from pCAG-tdTomato (Addgene 83029) by PCR, and a P2A sequence (a viral DNA element that has the ability to self-cleave) was added to the 3′ end. The resulting tdTomato-P2A construct was then fused in frame to the THRB2 start codon (illustrated in FIG. 3). The purpose of the CRR line was to track red/green cone photoreceptors and rod photoreceptors in real time during differentiation in live organoids. This line also enabled red/green cone cells and rod cells to be individually purified for bulk RNAseq to be performed on them at 2 different times (day 50 (D50) and day 100 (D100)).

hPSC-Retinal Organoid differentiation:

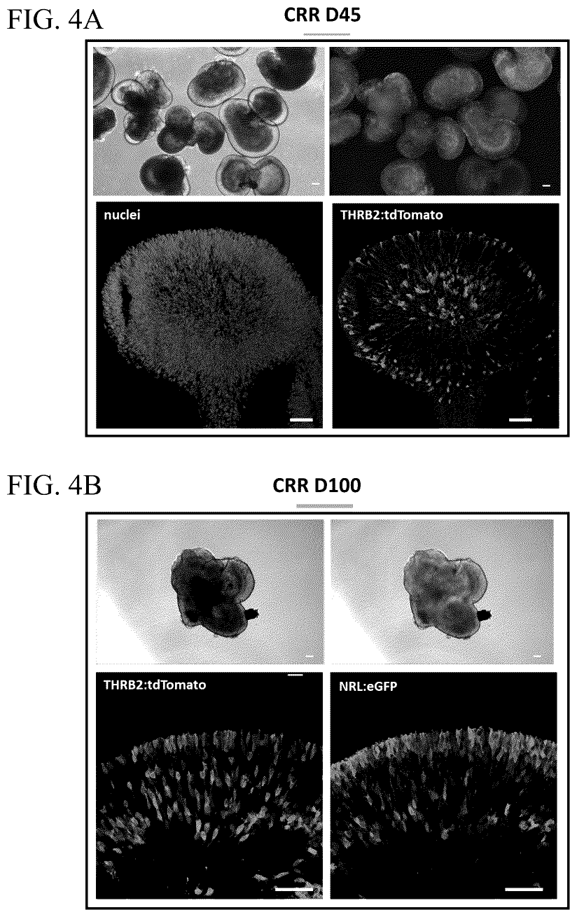

Stem cells from the CRR line were cultured and differentiated to retinal organoids using a previously published protocol (Capowski et al., 2019, Develop. 146: dev171868). Briefly, pluripotent stem cell colonies were detached from cell culture plates to form embryoid bodies (EBs). EBs were transitioned from mTeSR plus to a neural induction medium (NIM; DMEM:F12 1:1, 1% N2 supplement, 1×MEM nonessential amino acids (MEM NEAA), 1×GlutaMAX (Thermo Fisher) and 2 μg/ml heparin (Sigma)) over the course of 4 days. On day (D) 6, 0.75 nM BMP4 (R&D Systems) was added to fresh NIM and one day later, EBs were plated on Matrigel at a density of 200 EBs per well of a 6-well plate. Half the media was replaced with fresh NIM on D9, D12, and D15, and on D16 the media was changed to Retinal Differentiation Medium (RDM; DMEM:F12 3:1, 2% B27 supplement, MEM NEAA, 1× antibiotic, antimycotic (Thermo Fisher) and 1×GlutaMAX). On D25-D30, optic vesicle-like colonies became visually apparent by brightfield microscopy and were dissected with an MSP ophthalmic surgical knife (Surgical Specialties Corporation). The resulting free-floating retinal organoids were maintained in poly-HEMA-coated flasks (polyHEMA from Sigma) with twice-weekly feeding of 3D-RDM (DMEM:F12 3:1, 2% B27 supplement, 1×MEM NEAA, 1× antibiotic, anti-mycotic, and 1×GlutaMAX with 5% FBS, 100 μM taurine, 1:1000 chemically defined lipid supplement (11905031, Thermo Fisher)). Live retinal organoid cultures were imaged on a Nikon Ts2-FL microscope equipped with a DS-Fi3 camera or on a Nikon Ts100 microscope equipped with a QImaging CE CCD camera. The CRR retinal organoids began to demonstrate THRB2-tdTomato fluorescence in early red/green cone photoreceptor precursor cells around D40, and NRL-eGFP in rod photoreceptors around d70. FIG. 4A shows a D50 CRR retinal organoid with THRB2-tdTomato-positive CPP cells. FIG. 4B shows a D100 CRR retinal organoid with THRB2-tdTomato-positive red/green cone cells and NRL-eGFP-positive rod cells.

### Bulk RNA Sequencing and Data Processing

CRR retinal organoids were dissociated into single cells using papain (Worthington Biochemical papain dissociation system) and sorted on a BD FACSAria housed in a biosafety cabinet into (1) THRB2-tdTomato-negative (D50-N) non-photoreceptors and THRB2-tdTomato-positive red/green cone precursor cells (D50 Th-C) at Day (D) 50 (i.e., D50) and THRB2-tdTomato-negative/GFP-negative non-photoreceptors (D100-N), THRB2-tdTomato-positive red/green cone cells (D100 Th-C) and NRL-cGFP-positive rod cells (D100 R) at D100. RNA was isolated from each sample using the RNAeasy mini spin kit (Qiagen) according to the manufacturer's instructions and libraries for sequencing were prepared using the Illumina TruSeq Stranded Total RNA Library Prep kit for humans. Libraries were sequenced on a NovaSeq6000, and 30 million 2×150 nucleotide (nt) reads were collected for each sample. Bioinformatic analysis of transcriptomic data adhered to recommended ENCODE guidelines and best practices for RNA-Seq (Encode Consortium, 2016). Alignment of adapter-trimmed (Skewer v0.1.123; Jiang et al., 2014, Sci Rep 4:7175) 2×150 (paired-end; PE) bp strand-specific Illumina reads to the Homo sapiens GRCh38.p10 genome (assembly accession NCBI:GCA 000001405.25) was achieved with the Spliced Transcripts Alignment to a Reference (STAR v2.5.3a) software (Dobin et al., 2013, Bioinformatics 29:15-21). Expression estimation was performed with RSEM v1.3.0 (RNASeq by Expectation Maximization; Li and Dewey, 2011, BMC Bioinformatics 12:323). To test for differential gene expression among individual group contrasts, expected read counts obtained from RSEM were used as input into edgeR (v3.16.5; Robinson et al., 2010, Bioinformatics 26:139-40). Inter-sample normalization was achieved with the trimmed mean of M-values (TMM; Robinson and Oshlack, 2010, Genome Biol. 11: R25) method. Statistical significance of the negative-binomial regression test was adjusted with a Benjamini-Hochberg FDR correction at the 5% level (Reiner, Yekutieli, and Benjamini 2003, Bioinformatics 19:368-375). Prior to statistical analysis with edgeR, independent filtering was applied, and genes were required to have a count-per-million (CPM) above k in n samples, where k is determined by minimum read count (10 reads) and by the sample library sizes and n is determined by the number of biological replicates in each group. The validity of the Benjamini-Hochberg FDR multiple testing procedure was evaluated by inspection of the uncorrected p-value distribution.

### Example 2

### RNA Seq Analysis for Identifying Surface Markers

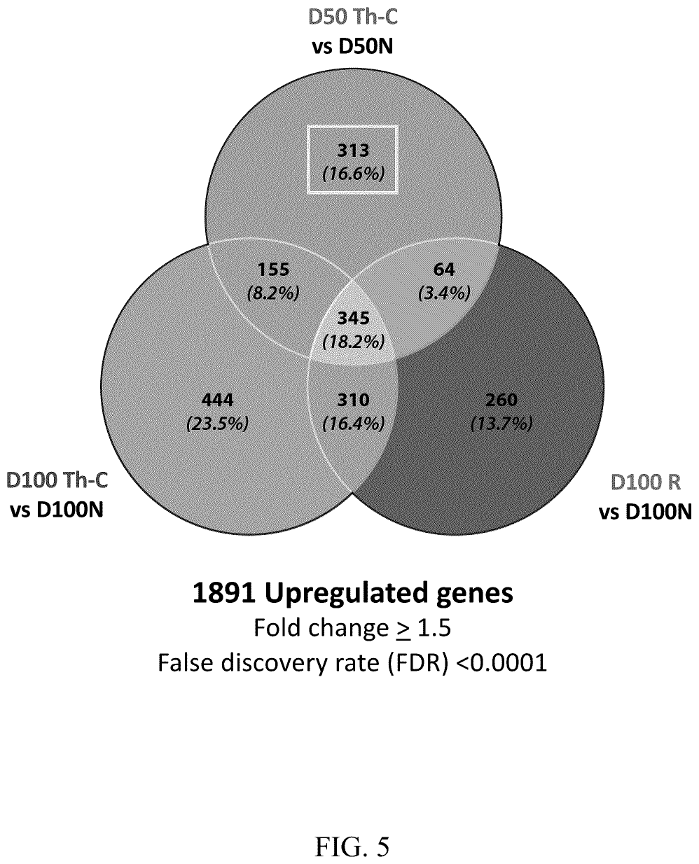

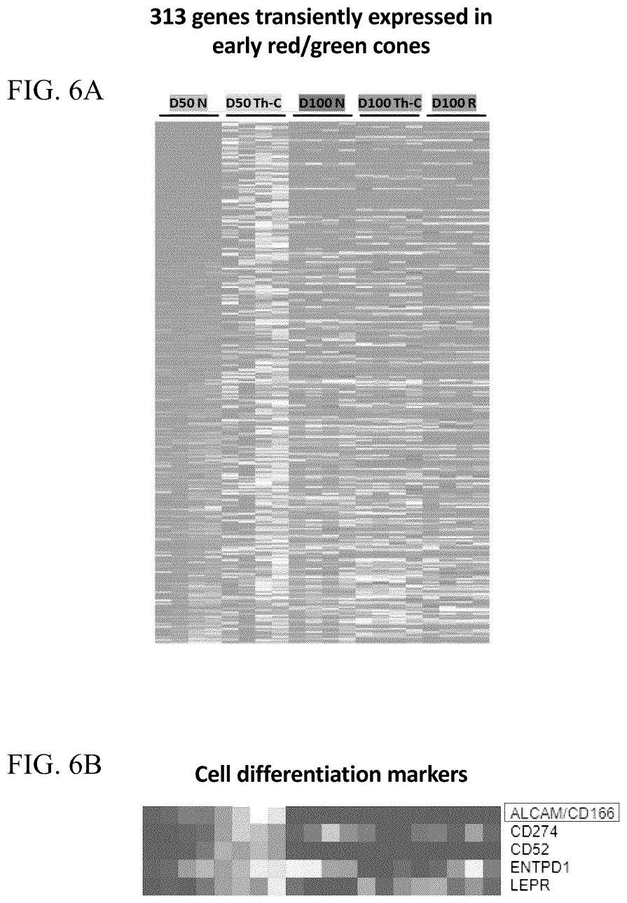

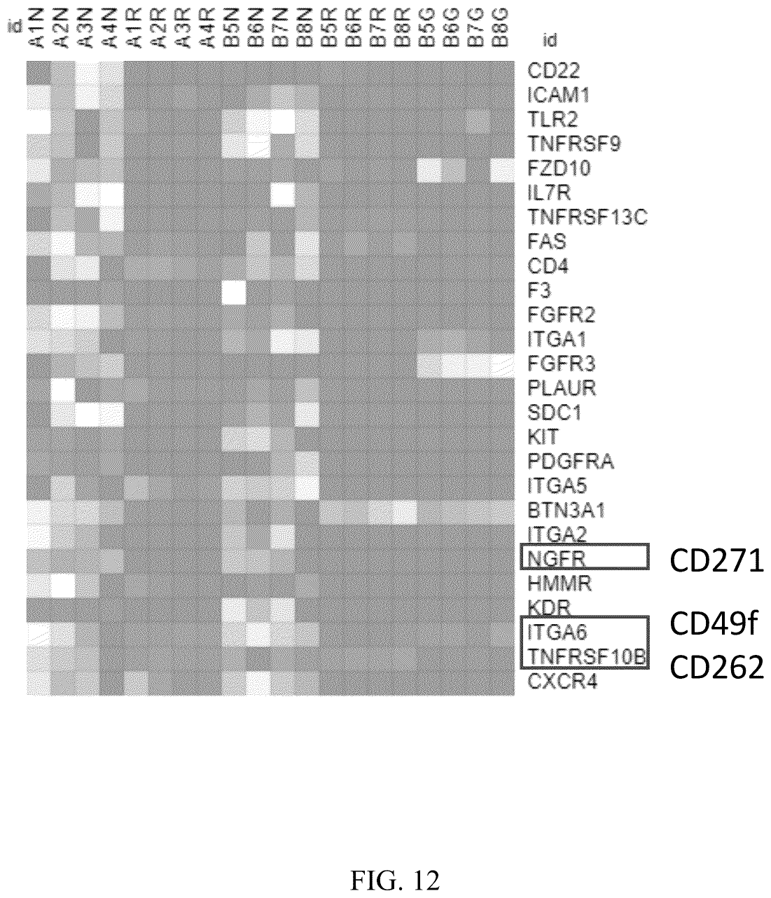

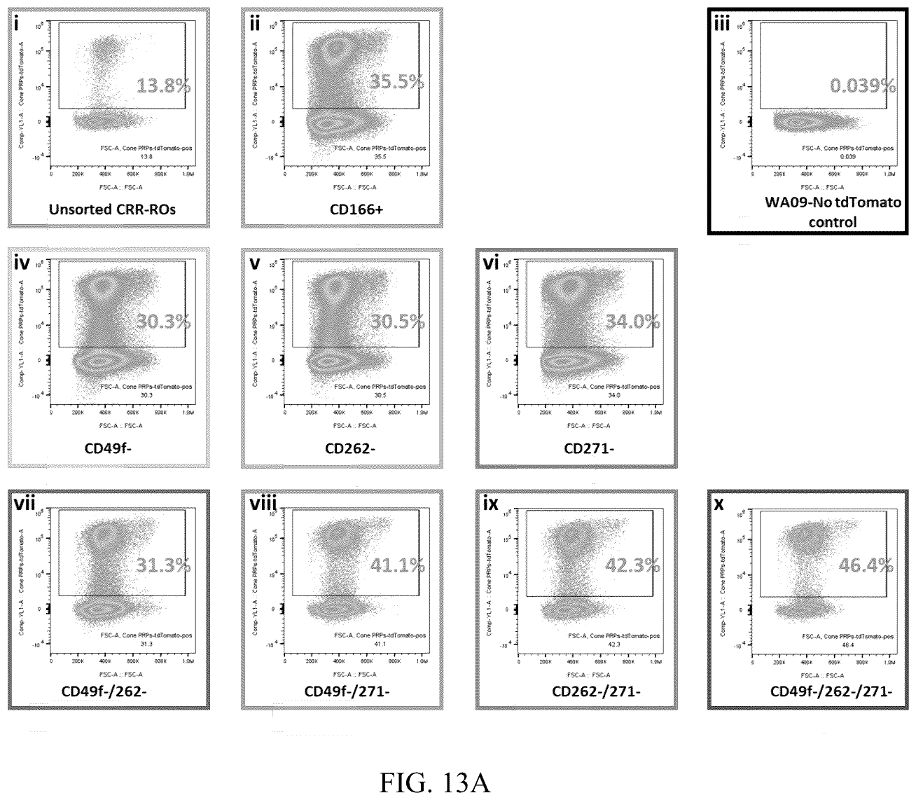

Lists of genes with significant differential expression between two given samples were obtained by retaining only genes with a minimum 1.5-fold change (either up- or down-regulated) and false discovery ratio (FDR) less than 0.0001. The 313 genes (illustrated in FIGS. 5 and 6A) that were upregulated only in D50 Th-C (red/green CPPs) were further analyzed by Gene Set Enrichment Analysis (GSEA) to identify gene families including cell differentiation (CD) markers. FIG. 6B shows CD markers (including ALCAM, also known as ALCAM/CD166) retrieved from the 313 genes. FIG. 12 shows the list of CD markers that can be used for negative selection of early red/green CPPs.

### Example 3

### Retinal Organoid (RO) Immunohistochemistry (IHC)

Retinal organoids were fixed in 4% paraformaldehyde (Electron Microscopy Sciences) at room temperature (RT) with gentle agitation for 1 h, washed with 1×PBS, and cryopreserved in 15% sucrose in PBS for 40 min, followed by equilibration in 30% sucrose for 40 min. Retina organoids were flash frozen immediately post-equilibration and then cryosectioned at a thickness of 15 μm. Cryosections were blocked in blocking solution (10% normal donkey serum, 5% BSA, and 0.5% Triton X-100 in 1×PBS) for 1 h at room temperature and incubated at 4° C. overnight with primary antibodies (e.g. anti-ALCAM/CD166, BioLegend-Catalog number: 343902) prepared in blocking solution. After incubation, cryosections were washed three times in phosphate buffered saline (PBS), then incubated for 30 min in the dark at room temperature with appropriate fluorophore-conjugated secondary antibodies prepared in blocking solution. Thereafter, the immunostained cryosections were washed three times in PBS, mounted in Prolong Gold Antifade with DAPI (Thermo Fisher Scientific), and imaged using a Nikon A1 laser scanning confocal microscope with NIS Elements AR 5.0 software.

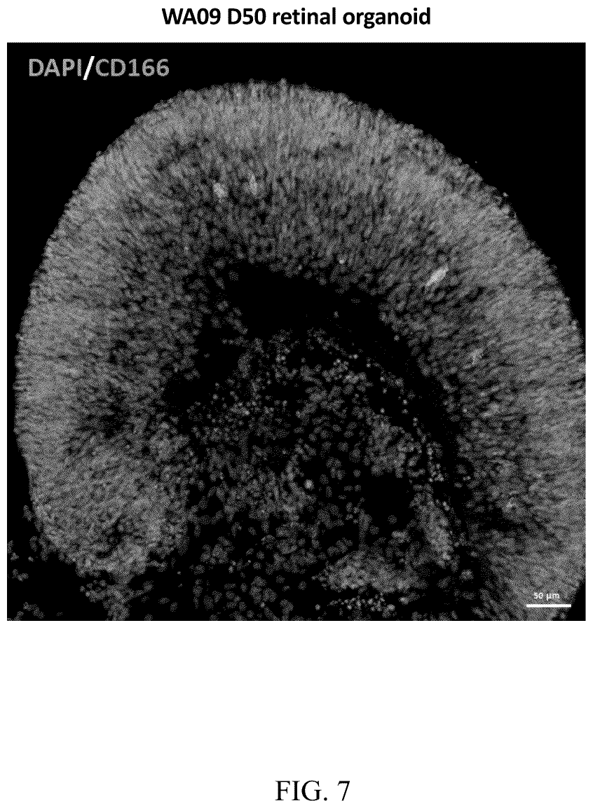

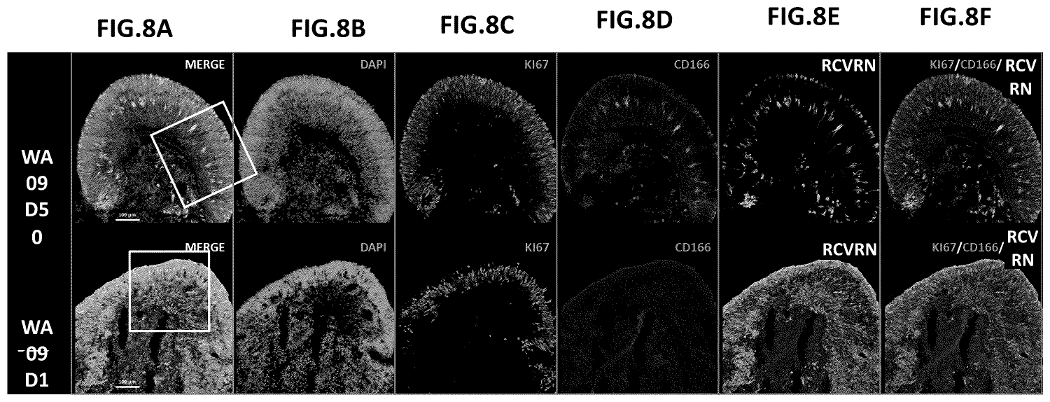

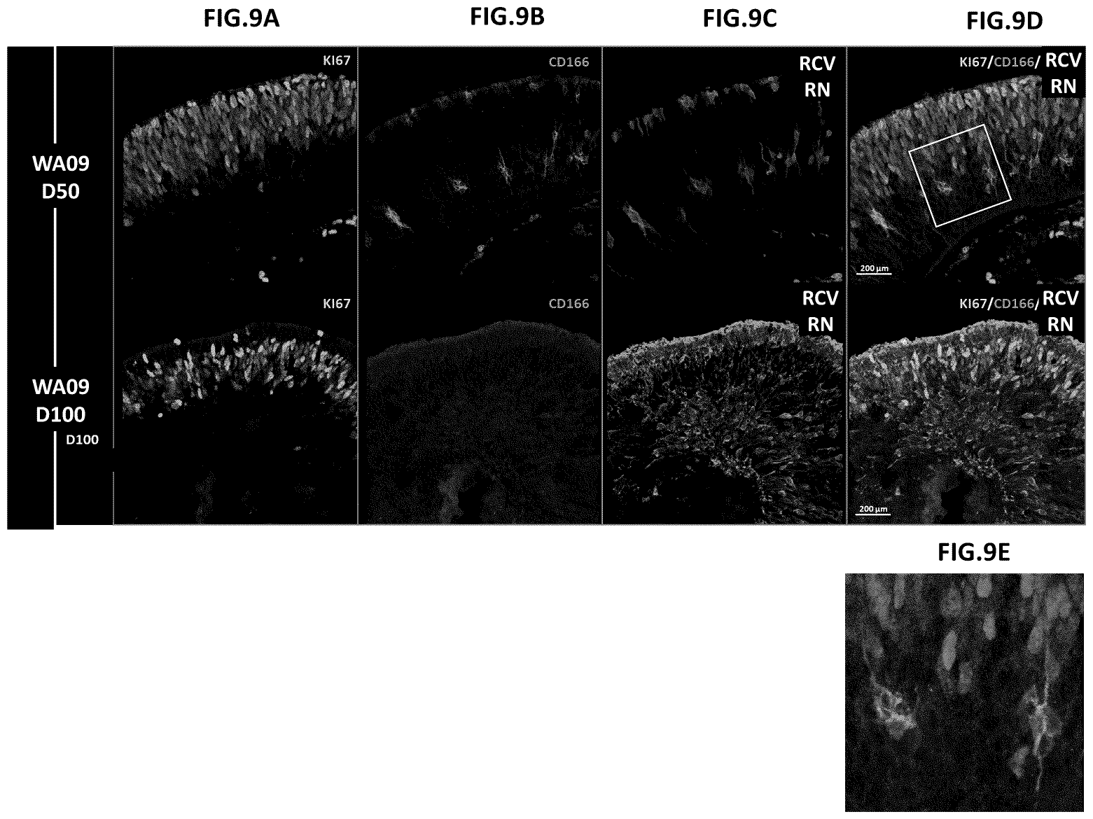

FIG. 7 shows ALCAM/CD166 expression in discrete cells of a D50 WA09 retinal organoid (i.e., an organoid without any fluorescence reporters). FIGS. 8 and 9 show the presence of ALCAM/CD166-positive cells in D50 retinal organoids, but not in D100 retinal organoids.

### Example 4

Using ALCAM/CD166 as a Selective Cell Surface Marker for Human Red/Green Cone Photoreceptor Precursor (CPP) Isolation (i.e. Positive MACS)

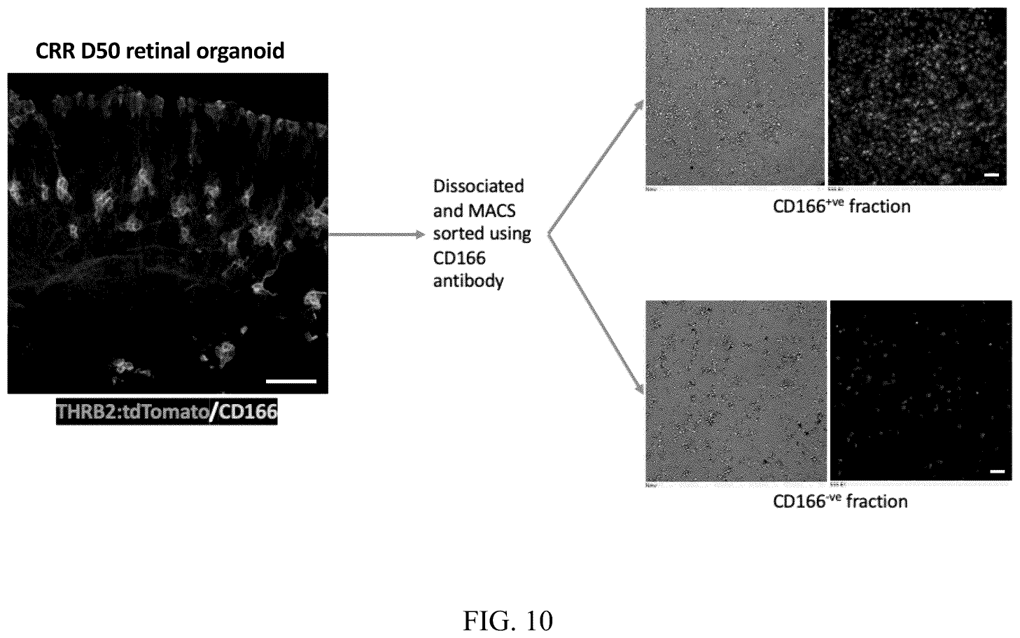

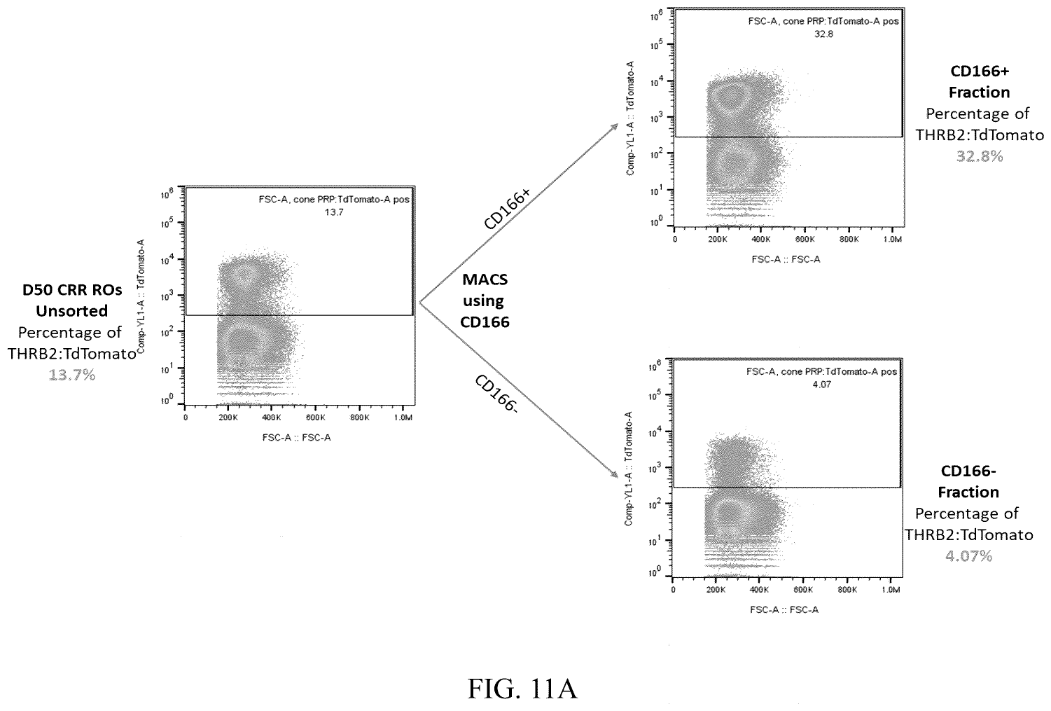

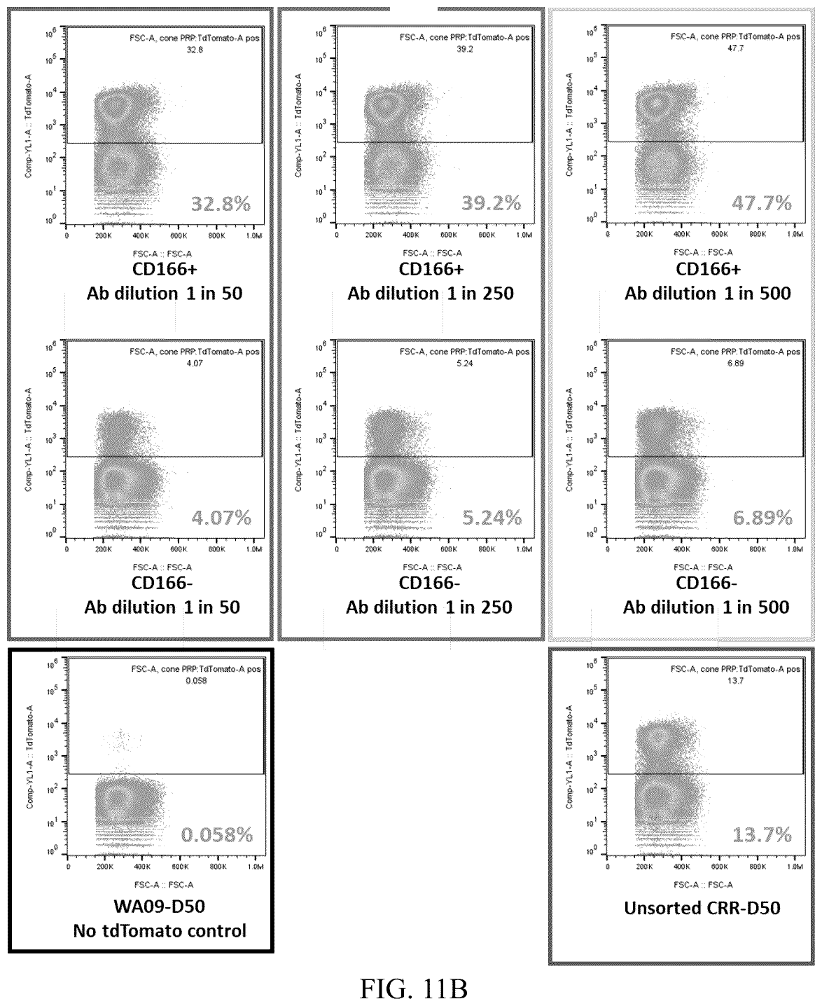

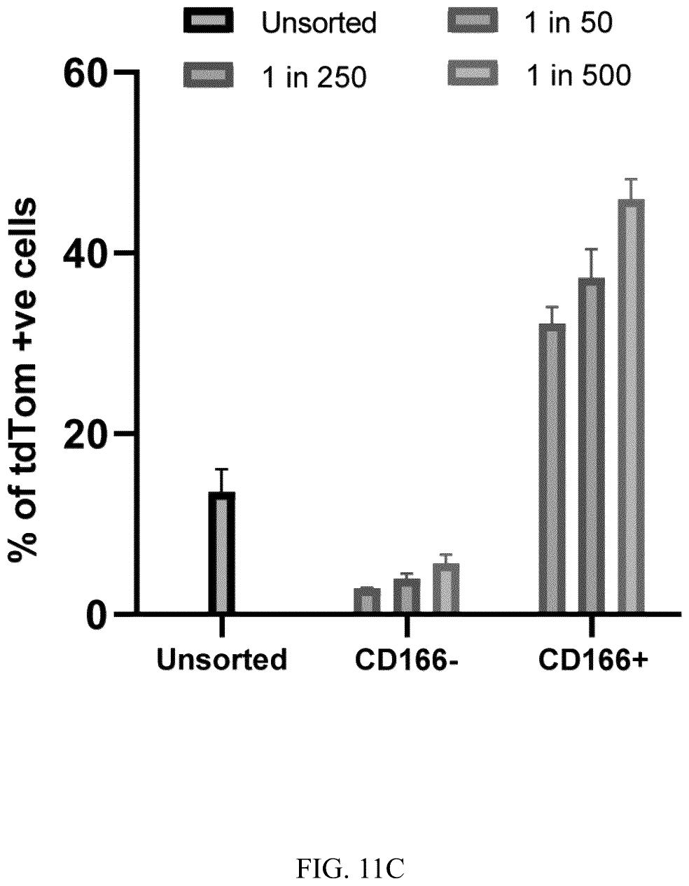

Co-localization of ALCAM/CD166 expression and tdTomato in D50 CRR retinal organoids was first confirmed by immunocytochemistry (ICC; see FIG. 10). Thereafter, D50 CRR retinal organoids were dissociated into single cells using 10× TrypLE select (Thermo Fisher). The dissociated cells were incubated with Biotin-conjugated ALCAM/CD166 (Miltenyi Biotec-Catalog number: 130-126-100) for 20 min at 4° C., then washed three times with MACS buffer with 0.5% bovine serum albumin (BSA; Miltenyi Biotec). Thereafter, cells were incubated with Anti-Biotin microbeads and passed through an LS column (Miltenyi Biotec) placed on a magnetic stand. The cells that flowed through the column were collected as the ALCAM/CD166-negative fraction. Thereafter, the column was removed from the magnetic stand and the remaining cells in the column were washed off to obtain the ALCAM/CD166-positive fraction. This is referred to as positive MACS sorting. The percentage of tdTomato-positive cells in the unsorted, ALCAM/CD166-positive and ALCAM/CD166-negative cell populations were then quantified using flow cytometry (shown in FIG. 11A-FIG. 11C).

### Example 5

### Negative MACS for Human Red/Green Cone Photoreceptor Precursor (CPP) Enrichment

Positive enrichment via MACS carries a potential disadvantage of having antibodies and magnetic particles bound to CPPs after sorting. An alternative approach is negative MACS in which unwanted cell types (non-CPPs) are magnetically labelled such that they remain in the magnetic column while the cells of interest (CPPs) flow through the magnetic column and are collected. Thus, the cells of interest (CPPs) separated by negative MACS are not bound with antibodies and magnetic particles, making them a more desirable source for human transplantation purposes. The cell surface proteins that were found to be present in non-CPPs are listed in FIG. 12. The cell surface antibodies we specifically chose to test for negative MACS are boxed in FIG. 12. D50-56 CRR retinal organoids were dissociated into single cells using 10×TrypLE select (Thermo Fisher). The dissociated cells were incubated with a single or a combination of the following Biotin-conjugated antibodies:
ITGA6/CD49f (Miltenyi Biotec—Catalog number: 130-123-243)—Dilution used—1 in 50
TNFRSF10B/CD262 (Miltenyi Biotec—Catalog number: 130-097-303)—Dilution used—1 in 11
NGFR/CD271 (Miltenyi Biotec—Catalog number: 130-112-608)—Dilution used—1 in 50

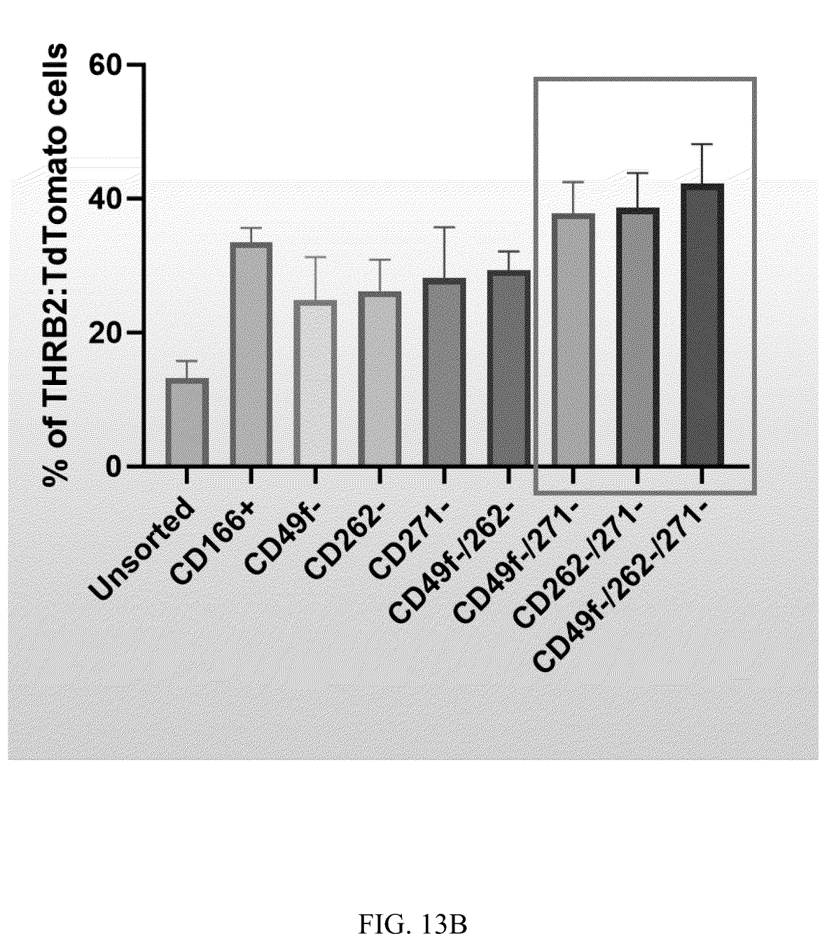

for 20 min at 4° C., then washed three times with MACS buffer with 0.5% bovine serum albumin (BSA; Miltenyi Biotec). Thereafter, cells were incubated with Anti-Biotin microbeads and passed through an LS column (Miltenyi Biotec) placed on a magnetic stand. The cells that flowed through the column were collected as the negative fraction. The percentage of tdTomato-positive cells in the unsorted, ALCAM/CD166-positive and ALCAM/CD166-negative cell populations were then quantified using flow cytometry (shown in FIG. 13A-B).

### Example 6

Formation of Microaggregates from ALCAM/CD166 Sorted CPPs

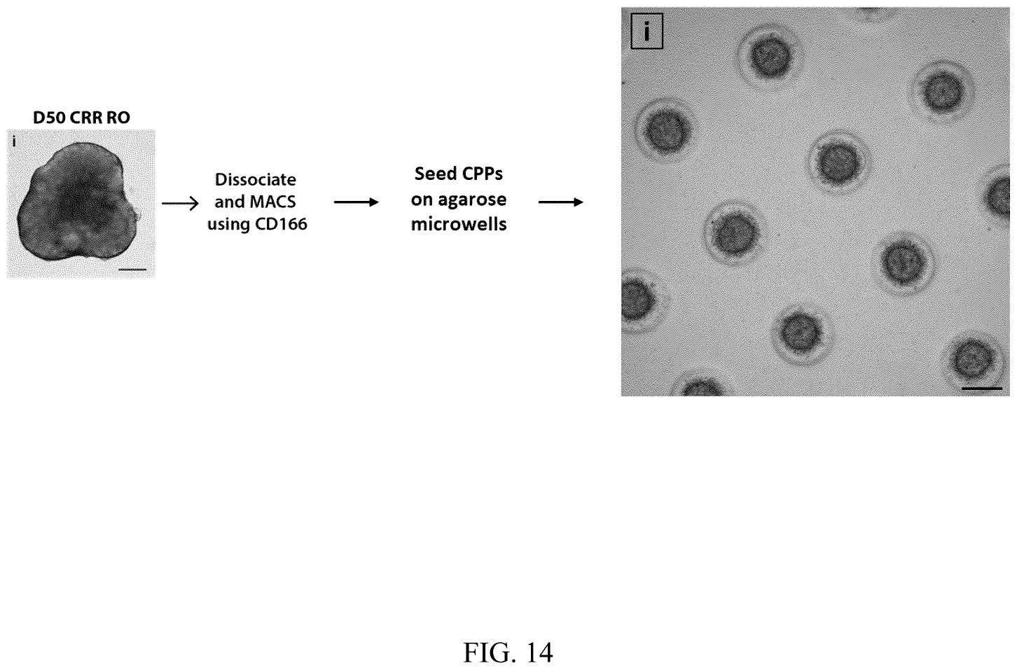

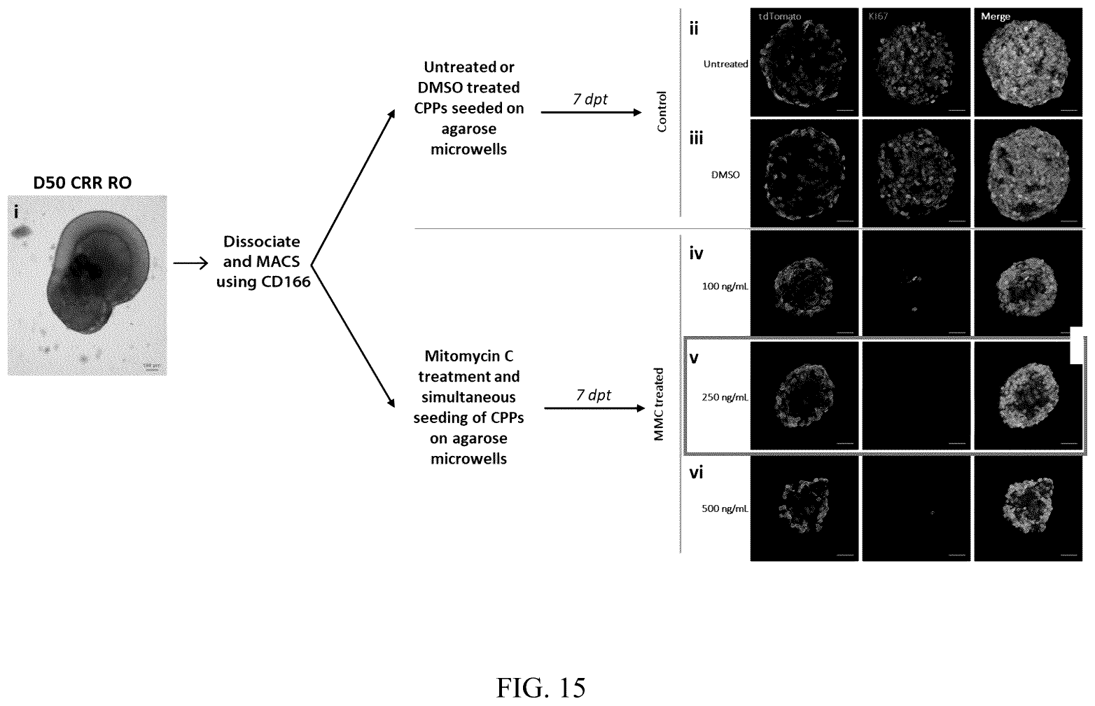

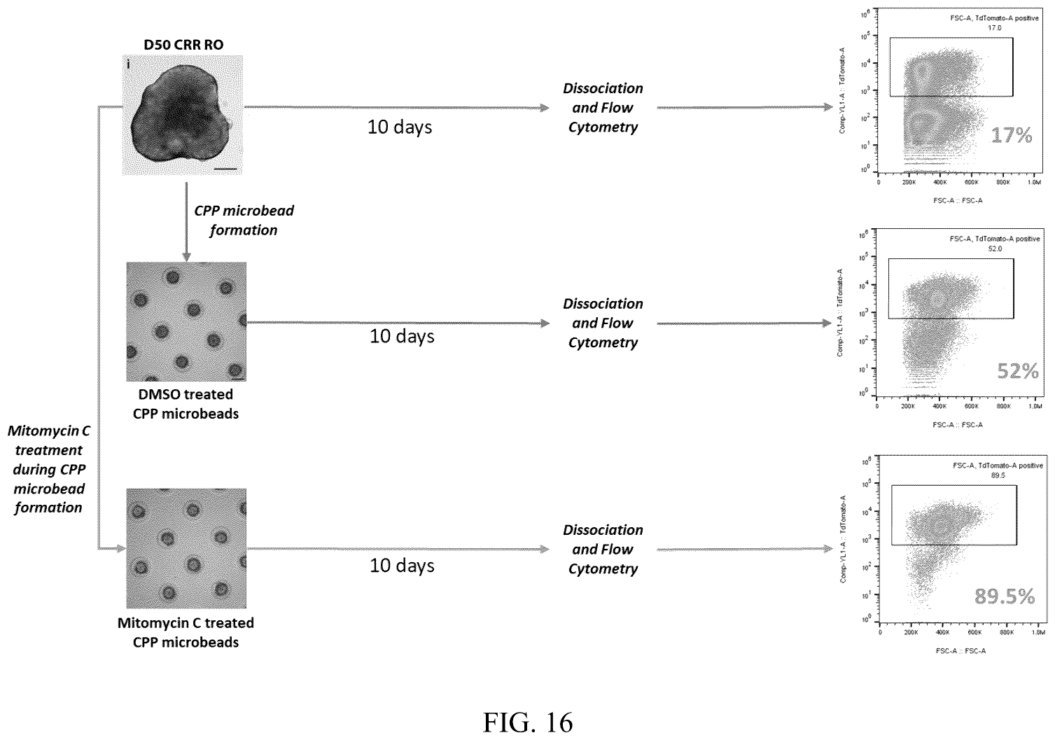

Preliminary transplantation experiments of fully dissociated ALCAM/CD166-sorted CPP single cell suspensions in rats resulted in high loss of cells due to reflux during cell injection. Thus, the option of culturing CPPs in the form of small aggregates, referred to as CPP microaggregates, was explored. For this purpose, agarose microwells were prepared using micromolds (microtissues, inc.) according to manufacturer's instructions. ALCAM/CD166-positive sorted CPPs from D50 CRR/WA09 ROs resuspended in 3D-RDM were seeded on agarose microwells (FIG. 14). The seeding density varied between 250-8000 cells/microwell. After 24 hours, the CPPs on agarose microwells came together to form microspheroids called as CPP microaggregates. These CPP microaggregates were maintained on agarose microwells for 7-10 days before proceeding with immunohistochemistry (FIG. 15) or flow cytometry (FIG. 16). The CPP microaggregates can be maintained on agarose microwells for up to 6 months (FIG. 14 i).

Both untreated and DMSO control-treated CPP microaggregates grew in size due to proliferation as indicated by Ki-67 immunostaining (FIG. 15.ii-iii). There were few Ki-67 positive cells in CPP microaggregates treated with 100 ng/mL MMC (FIG. 15.iv), whereas Ki-67 positive cells in CPP microaggregates treated with 250 and 500 ng/mL MMC were almost nil at 7 dpt. There was noticeable disruption in the morphology of CPP microaggregates treated with 500 ng/mL MMC. Due to the disruption, 250 ng/mL was chosen as the ideal concentration for further experiments.

The method disclosed herein for the formation of CPP microaggregates can be applied to both positive- and negative-sorted cells.

### Example 7

### Mitomycin C (MMC) Treatment of ALCAM/CD166 Sorted CPPs

Immunohistochemistry of D7 CPP microaggregates (7 days post bead formation) revealed the presence of proliferating cells marked by the expression of Ki-67 (FIG. 15 ii). The presence of proliferating cells is not preferred for transplantation as they proliferate significantly, forming large, haphazard transplant grafts with a progressively lower percentage of CPPs. In order to minimize proliferation of non-CPPs and maximize localization and overall percentage of CPPs in subretinal transplants, the CD166 sorted CPPs were treated with mitomycin C (MMC), a cell proliferation inhibitor. The CPP microaggregates were maintained in 3D-RDM containing mitomycin C for 24-72 hours after which the CPP microaggregates were washed with 3D-RDM and maintained in 3D-RDM for an additional 4-11 days prior to immunohistochemistry (FIG. 15 iv-vi) or flow cytometry (FIG. 16). It can be noted that CPP microaggregates formation combined with MMC treatment increased the percentage of THRB2:tdTomato-positive red/green CPP cells from 17% to 89.5%. Both positive- and negative-sorted CPP cells and microaggregates can be treated with mitomycin C to increase the CPP percentage.

### Example 8

Transplantation of MMC-Treated or Untreated CPP Microaggregates into S334ter Rat Retina at 2 Months of Age

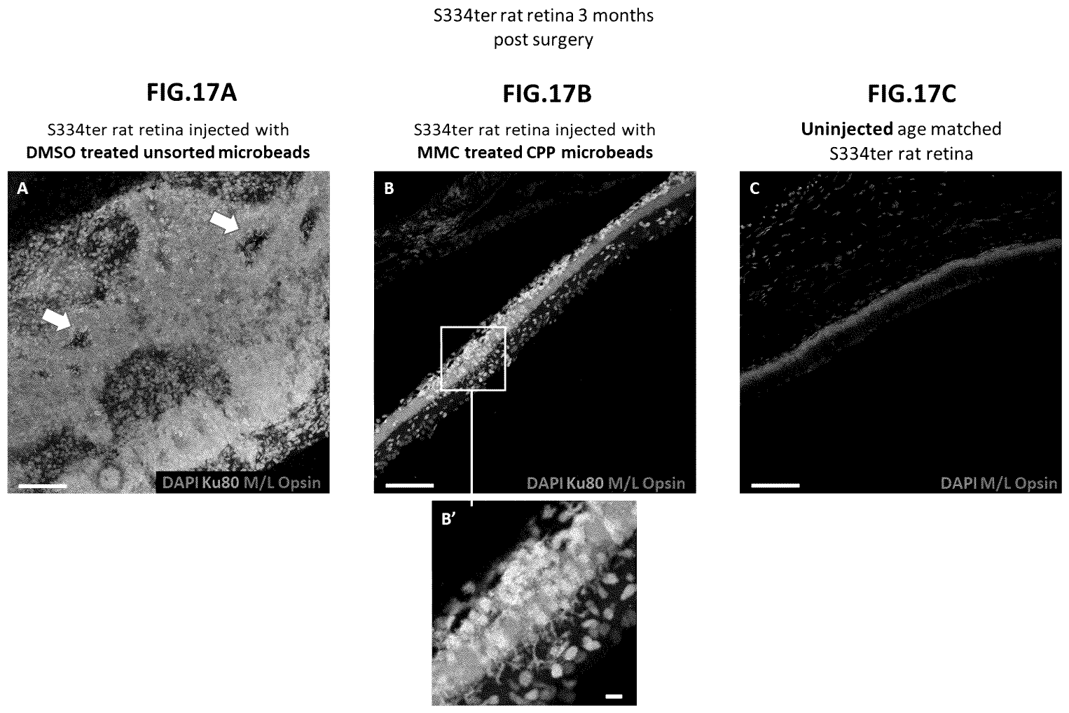

To evaluate the ability of CPP microaggregates to survive and mature into desired red/green cone cells in the host retina, CPP microaggregates along with respective controls were transplanted into the sub-retinal space of S334ter rats. This immunocompromised rat model has a rhodopsin mutation that leads to rapid rod photoreceptor loss and progressive cone loss, similar to patients with retinitis pigmentosa. For the purpose of transplantation, beads from WA09 non fluorescent cell line were produced. Microaggregates from two conditions, 1) unsorted/DMSO treated (control), and 2) CD166-positive sort/MMC250-treated, were collected from the microwells and resuspended in balanced salt solution (BSS) at a concentration of 800 beads per microliter (250 cells per microaggregate and thus ˜200,000 cells per microliter). S334ter rats (n=3) were anesthetized and transplanted in the sub-retinal space (SRS) with 2 ul of microaggregate suspension from each of the conditions per eye. The animals were sacrificed 3 months post-surgery (5 months of age) for IHC analysis (FIG. 17).

FIG. 17A shows that S334ter rat retina transplanted with DMSO control-treated unsorted microaggregates had a significant expansion of grafted human cells marked by human nuclear marker Ku80 (red). The graft contained neural rosettes (white arrows) that predominantly contained rod photoreceptors (data not shown) and few red/green cone photoreceptors, marked by the presence of few M/L Opsin+ cells (green). The graft also contained a majority of Ki67+ proliferative cells and retinal progenitors (data not shown). FIG. 17B shows that S334ter rat retina transplanted with MMC treated CPP microaggregates had very little expansion of transplanted human cells (Ku-80 in red), and primarily consisted of cones. The graft did not form neural rosettes and showed enhanced integration of transplanted human cells (Ku-80 in red) in the host retina (DAPI-positive/Ku80 negative cells). The transplanted cells predominantly developed into mature red/green cones marked by the expression of M/L Opsin. (see FIG. 17B′—magnified image). There were very few rod photoreceptors (data not shown). FIG. 17C shows that the uninjected age matched (5 months old) S334ter rat retina is thinned due to the loss of outer nuclear layer (i.e., the layer containing photoreceptors) from the underlying photoreceptor degenerative disease caused by the mutation in Rhodopsin. M/L Opsin immunostaining in uninjected control retinas showed the near complete absence of host M/L cone photoreceptors in 5-month-old S334ter rats (only a few disorganized cones remain at this time).

All publications, patents, and patent applications mentioned in this specification are herein incorporated by reference to the same extent as if each individual publication, patent, and patent application was specifically and individually indicated to be incorporated by reference.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings the disclosure, and the appended claims. The mere fact that certain measures or features are recited in mutually different dependent claims does not indicate that the combination of these measures or features cannot be used. Any reference signs in the claims should not be construed as limiting the scope.

Sequence Listing
Endogenous sequence encompassing intron1/
exon2 junction of NRL gene (lowercase-
intron1, UPPERCASE-EXON2, Translation
Start Site (TSS) underlined):
(SEQ ID NO: 1)
aagggaaaacgggtcagaagggtgaaggtgatctgccaagttcacacaac
gtaacaagttctgaatagaaggaaaatcaatcttggtctgttccccttgg
agagactggccttgaggaaagatggtggccagttgattctgatctttcta
gaactaattctaggacctttcttttttccatataaagcctcttgccccta
caaaagggattcattgattgattaatccatagaacaaatgtacactgagg
gtcttctataggcaaggcacttccctgagtgctttgagagacagagagct
ataagaacacattatccttggctcttaaaattcacagcatacatgccttc
ccactgtcctaaaatatacactcactttgtcacagttaattaagcaattt
aaaaaaatggcttggaaatggaatgatgcctcttgagatgacagacctct
cggcatgtctcagcactagggttgggagcactgccatactgctcttgctt
ttcatagatcctgaggcatcagtggggcgagaggctgtgctgtcctcttc
ctccttcaggaattcagctgcttgtcccctgtcaggagcccctgccctct
gaaaggttactcttcagcctggtggggactctgcagtgaacagagctgca
ccatccctctggctttcccaaactcttgctccagggcacttgggctttga
gggaagagggacttggtgaagaggggatggcaggtggcctccatgtgctc
cagacctctcctcctctttgcagGTGCACTCCTCCCAGCCCAGCTCCAGA
ATGGCCCTGCCCCCCAGCCCCCTGGCCATGGAATATGTCAATGACTTTGA
CTTGATGAAGTTTGAGGTAAAGCGGGAACCCTCTGAGGGCCGACCTGGCC
CCCCTACAGCCTCACTGGGCTCCACACCTTACAGCTCAGTGCCTCCTTCA
CCCACCTTCAGTGAACCAGGCATGGTGGGGGCAACCGAGGGCACCCGGCC
AGGCCTGGAGGAGCTGTACTGGCTGGCTACCCTGCAGCAGCAGCTGGGGG
CTGGGGAGGCATTGGGGCTGAGTCCTGAAGAGGCCATGGAGCTGCTGCAG
GGTCAGGGCCCAGTCCCTGTTGATGGGCCCCATGGCTACTACCCAGGGAG
CCCAGAGGAGACAGGAGCCCAGCACGTCCAG
eGFP sequence
(SEQ ID NO: 2):
ATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAGG
GCGAGGGCGATGCCACCTACGGCAAGCTGACCCTGAAGTTCATCTGCACC
ACCGGCAAGCTGCCCGTGCCCTGGCCCACCCTCGTGACCACCCTGACCTA
CGGCGTGCAGTGCTTCAGCCGCTACCCCGACCACATGAAGCAGCACGACT
TCTTCAAGTCCGCCATGCCCGAAGGCTACGTCCAGGAGCGCACCATCTTC
TTCAAGGACGACGGCAACTACAAGACCCGCGCCGAGGTGAAGTTCGAGGG
CGACACCCTGGTGAACCGCATCGAGCTGAAGGGCATCGACTTCAAGGAGG
ACGGCAACATCCTGGGGCACAAGCTGGAGTACAACTACAACAGCCACAAC
GTCTATATCATGGCCGACAAGCAGAAGAACGGCATCAAGGTGAACTTCAA
GATCCGCCACAACATCGAGGACGGCAGCGTGCAGCTCGCCGACCACTACC
AGCAGAACACCCCCATCGGCGACGGCCCCGTGCTGCTGCCCGACAACCAC
TACCTGAGCACCCAGTCCGCCCTGAGCAAAGACCCCAACGAGAAGCGCGA
TCACATGGTCCTGCTGGAGTTCGTGACCGCCGCCGGGATCACTCTCGGCA
TGGACGAGCTGTACAAGTAA
Endogenous sequence encompassing 5′UTR/
exon1 junction of THRB2 gene (lowercase-
5′UTR, UPPERCASE-EXON1, Transcription
Start Site (TSS) underlined):
(SEQ ID NO: 3)
attcaacaacagattcaaccaccaatatttgcacagctagattaatgttt
tggagtcagtcagtcatcaggctcattactactggattttctctggttca
tgctaataaactagattattgattttaggattttctttgtatccaaggat
attctctaagatgtctttataatgtttcatgcttaagctgtcaaccaaaa
tgacataacaatgagtaatcgttttataatcatgttgaaaggggttaaaa
acagcatataaaatgcatcttagcagcttcagagtaacattctcgctgtc
acttccctatctgggtggaggtcattcctacctgcctgccatcttcccca
ggaattaactagctgcatgcacagcttaagacctttgatagtcataaatc
tgctttccaatatgtttggatggCAGAAGAGCTCAAAAGACACCTCGGTC
TAAATAGGATTTTAGCACATCAGGTGCTATTACAAAGTAAAGCAGGCTGG
GTTGCTGTGTTGGTGGTGGTGTTTATTCATCTTCCCTTCGCGTTTCATGT
GTATGTATGTATGCTTCTCTGCGTATATGCCCAGCTCTCAGGGTGTATGG
GGCTGGAGAATGCATGCGTAGACTGTGTGTATATGTAAACTAGAAACTGA
ACCAGGGAAACAAAATGAACTACTGTATGCAAGAAATATATGAAGTGCAC
CCAGCTGCCGGCAGCAATTGCTACATGCAGTCCACTGATTATTACGCGTA
TTTTGAAGATAGTCCGGGTTACAGCGGTTGCGATGCTCAGGCTGTGCCCA
GTAACAACATATATATGGAACAGGCCTGGGCAGTGAATCAGCCTTATACC
TGTAGTTACCCTGGAAACATGTTTAAAAGCAAGGACTCTGACTTGGACAT
GGCCCTGAATCAATACAGCCAACCTGAATATTTCACTGAAGAAAAGCCTA
CTTTTTCTCAAGTGCAGTCGCCATCGTATTCTCAAAAAAAAG
tdTomato sequence
(SEQ ID NO: 4):
ATGGTGAGCAAGGGCGAGGAGGTCATCAAAGAGTTCATGCGCTTCAAGGT
GCGCATGGAGGGCTCCATGAACGGCCACGAGTTCGAGATCGAGGGCGAGG
GCGAGGGCCGCCCCTACGAGGGCACCCAGACCGCCAAGCTGAAGGTGACC
AAGGGCGGCCCCCTGCCCTTCGCCTGGGACATCCTGTCCCCCCAGTTCAT
GTACGGCTCCAAGGCGTACGTGAAGCACCCCGCCGACATCCCCGATTACA
AGAAGCTGTCCTTCCCCGAGGGCTTCAAGTGGGAGCGCGTGATGAACTTC
GAGGACGGCGGTCTGGTGACCGTGACCCAGGACTCCTCCCTGCAGGACGG
CACGCTGATCTACAAGGTGAAGATGCGCGGCACCAACTTCCCCCCCGACG
GCCCCGTAATGCAGAAGAAGACCATGGGCTGGGAGGCCTCCACCGAGCGC
CTGTACCCCCGCGACGGCGTGCTGAAGGGCGAGATCCACCAGGCCCTGAA
GCTGAAGGACGGCGGCCACTACCTGGTGGAGTTCAAGACCATCTACATGG
CCAAGAAGCCCGTGCAACTGCCCGGCTACTACTACGTGGACACCAAGCTG
GACATCACCTCCCACAACGAGGACTACACCATCGTGGAACAGTACGAGCG
CTCCGAGGGCCGCCACCACCTGTTCCTGGGGCATGGCACCGGCAGCACCG
GCAGCGGCAGCTCCGGCACCGCCTCCTCCGAGGACAACAACATGGCCGTC
ATCAAAGAGTTCATGCGCTTCAAGGTGCGCATGGAGGGCTCCATGAACGG
CCACGAGTTCGAGATCGAGGGCGAGGGCGAGGGCCGCCCCTACGAGGGCA
CCCAGACCGCCAAGCTGAAGGTGACCAAGGGCGGCCCCCTGCCCTTCGCC
TGGGACATCCTGTCCCCCCAGTTCATGTACGGCTCCAAGGCGTACGTGAA
GCACCCCGCCGACATCCCCGATTACAAGAAGCTGTCCTTCCCCGAGGGCT
TCAAGTGGGAGCGCGTGATGAACTTCGAGGACGGCGGTCTGGTGACCGTG
ACCCAGGACTCCTCCCTGCAGGACGGCACGCTGATCTACAAGGTGAAGAT
GCGCGGCACCAACTTCCCCCCCGACGGCCCCGTAATGCAGAAGAAGACCA
TGGGCTGGGAGGCCTCCACCGAGCGCCTGTACCCCCGCGACGGCGTGCTG
AAGGGCGAGATCCACCAGGCCCTGAAGCTGAAGGACGGCGGCCACTACCT
GGTGGAGTTCAAGACCATCTACATGGCCAAGAAGCCCGTGCAACTGCCCG
GCTACTACTACGTGGACACCAAGCTGGACATCACCTCCCACAACGAGGAC
TACACCATCGTGGAACAGTACGAGCGCTCCGAGGGCCGCCACCACCTGTT
CCTGTACGGCATGGACGAGCTGTACAAG

1. A method for obtaining an enriched population of human red/green cone photoreceptor precursor (CPP) cells, the method comprising sorting a mixed population of retinal cells contacted with a detectably labeled binding agent that specifically binds ALCAM/CD166 expressed on the cell surface.

2. The method of claim 1, wherein the cells are contacted with the detectably labeled specific binding agent for ALCAM/CD166 before or during sorting.

3. The method of claim 2, wherein the specific binding agent is an antibody or antigen-binding fragment thereof that specifically binds ALCAM/CD166.

4. The method of claim 2, wherein ALCAM/CD166 positive cells are selected by a using a cell surface marker sorting technique.

5. The method of claim 2, wherein the detectable label is a fluorescent label.

6. The method of claim 5, wherein the cells are sorted by fluorescence-activated cell sorting (FACS).

7. The method of claim 2, wherein the detectable label is a magnetic label.

8. The method of claim 7, wherein the cells are sorted by magnetic-activated cell sorting (MACS).

9-28. (canceled)

29. A method of obtaining an enriched population of red/green CPP cells, the method comprising sorting a mixed population of retinal cells contacted with one or more detectably labeled binding agents each of which specifically binds ITGA6/CD49f, TNFRSF10B/CD262, NGFR/CD271, or combinations thereof, expressed on the cell surface, and sorting to obtain any of ITGA6/CD49f-negative, TNFRSF10B/CD262-negative, NGFR/CD271-negative cells, or combinations thereof.

30. The method of claim 29, wherein the specific binding agent is an antibody or antigen-binding fragment thereof each of which specifically binds ITGA6/CD49f, TNFRSF10B/CD262, NGFR/CD271, or combinations thereof.

31. The method of claim 30, wherein the antibody is one or more biotin-conjugated antibodies.

32. The method of claim 31, wherein the biotin-conjugated antibody specifically binds one or more of ITGA6/CD49f, TNFRSF10B/CD262, NGFR/CD271, or combinations thereof.

33. The method of claim 32, wherein the cells are incubated with the one or more biotin-conjugated antibodies.

34. The method of claim 33, wherein the cells are further incubated with anti-biotin microbeads.

35. The method of claim 34, wherein the cells are sorted by magnetic-activated cell sorting (MACS).

36. The method of claim 35, wherein ITGA6/CD49f-negative, TNFRSF10B/CD262-negative, NGFR/CD271-negative cells, or combinations thereof are separated from ITGA6/CD49f-positive, TNFRSF10B/CD262-positive, or NGFR/CD271-positive cells.

37-46. (canceled)

47. A method of obtaining CPP microaggregates the method comprising treating a population of CPP cells with a cell proliferation inhibitor, wherein the cells are produced by obtaining microaggregates from a population of CPP cells, wherein the cells are obtained by sorting a mixed population of retinal cells contacted with a detectably labeled binding agent that specifically binds ALCAM/CD166 expressed on the cell surface, and seeding at least one of ALCAM/CD166-positive cells wherein the cells are seeded at a density for at least 24 hours, and; incubating CPP microaggregates in 3D-Retinal Differentiation Medium (RDM) containing the cell proliferation inhibitor for an amount of time, wherein cells are washed with 3D-RDM after incubation.

48. The method of claim 47, wherein the cell proliferation inhibitor is mitomycin C.

49. The method of claim 47, wherein the CPP microaggregates are incubated for 24-72 hours.

50. A method of obtaining CPP microaggregates, the method comprising treating a population of CPP cells with a cell proliferation inhibitor, wherein the cells are obtained by sorting a mixed population of retinal cells comprising ALCAM/CD166-positive CPP cells contacted with one or more detectably labeled binding agents each of which specifically binds ITGA6/CD49f, TNFRSF10B/CD262, NGFR/CD271, or combinations thereof, expressed on the cell surface, and sorting to obtain any of ITGA6/CD49f-negative, TNFRSF10B/CD262-negative, NGFR/CD271-negative cells, or combinations thereof, and seeding at least one of ITGA6/CD49f-negative, TNFRSF10B/CD262-negative, NGFR/CD271-negative cells, or combinations thereof wherein the cells are seeded at a density for at least 24 hours; and incubating CPP microaggregates in 3D-RDM containing the cell proliferation inhibitor for an amount of time, wherein cells are washed with 3D-RDM after incubation.

51. The method of claim 50, wherein the cell proliferation inhibitor is mitomycin C.

52. The method of claim 50, wherein the CPP microaggregates are incubated for 24-72 hours.
